(19) United States
(12) Patent Application Publication
(10) Pub. No.: US 2026/0345008 A1
(43) Pub. Date: Nov. 5, 2026

(54) IMAGING SYSTEM

(71) Applicant: Gentuity, LLC, Sudbury, MA (US)

(72) Inventors: Christopher C. Petroff, Groton, MA (US); Andrea Rivas, Natick, MA (US); Christopher L. Petersen, Willowick, OH (US); Christopher Battles, Seymour, CT (US); Nareak Douk, Lowell, MA (US); Andrew Belle-Isle, Lowell, MA (US)

(21) Appl. No.: 19/118,881

(22) PCT Filed: Oct. 13, 2023
(86) PCT No.: PCT/US2023/035132

§ 371 Date: Apr. 7, 2025

Related U.S. Application Data
(60) Provisional application No. 63/416,170, filed on Oct. 14, 2022.
(60) Provisional application No. 63/532,223, filed on Aug. 11, 2023.
(60) Provisional application No. 63/542,279, filed on Oct. 3, 2023.

(51) Int. Cl. A61B 5/00

(52) U.S. Cl. CPC A61B 5/0066; A61B 5/0084; A61B 5/6852

(57) ABSTRACT

Provided herein are imaging systems for a patient including an imaging probe and an imaging assembly. The imaging probe includes: a first elongate shaft; a second elongate shaft a positioned within a lumen of the first elongate shaft; a rotatable optical core positioned within a lumen of the second elongate shaft; and an optical assembly positioned proximate a distal end of the rotatable optical core. The optical assembly can direct light to tissue to be imaged and to collect reflected light from the tissue to be imaged. The imaging assembly can emit light into the imaging probe and receive the reflected light collected by the optical assembly.

### RELATED APPLICATIONS

This application claims benefit of U.S. Provisional Application Ser. No. 63/416,170 (Docket No. GTY-023-PR1), titled “Imaging System”, filed Oct. 14, 2022, the content of which is incorporated by reference in its entirety.

This application claims the benefit of U.S. Provisional Application Ser. No. 63/532,223 (Docket No. GTY-025-PR1), titled “Enhanced Imaging System”, filed Aug. 11, 2023, the content of which is incorporated by reference in its entirety.

This application claims the benefit of U.S. Provisional Application Ser. No. 63/542,279 (Docket No. GTY-026-PR1), titled “Imaging System”, filed Oct. 3, 2023, the content of which is incorporated by reference in its entirety.

This application is related to U.S. Provisional Application Ser. No. 62/148,355 (Docket No.: GTY-001-PR1), titled “Micro-Optic Probes for Neurology”, filed Apr. 16, 2015, the content of which is incorporated by reference in its entirety.

This application is related to U.S. Provisional Application Ser. No. 62/322,182 (Docket No. GTY-001-PR2), titled “Micro-Optic Probes for Neurology”, filed Apr. 13, 2016, the content of which is incorporated by reference in its entirety.

This application is related to International PCT Patent Application Serial Number PCT/US2016/027764 (Docket No. GTY-001-PCT), titled “Micro-Optic Probes for Neurology” filed Apr. 15, 2016, Publication Number WO 2016/168605, published Oct. 20, 2016, the content of which is incorporated by reference in its entirety.

This application is related to U.S. patent application Ser. No. 15/566,041 (Docket No. GTY-001-US), titled “Micro-Optic Probes for Neurology”, filed Oct. 12, 2017, U.S. Pat. No. 11,278,206 , issued Mar. 22, 2022, the content of which is incorporated by reference in its entirety.

This application is related to U.S. patent application Ser. No. 17/668,757 (Docket No. GTY-001-US-CON1), titled “Micro Optic Probes for Neurology”, filed Feb. 10, 2022, United States Publication Number 2022-0218206, published Jul. 14, 2022, the content of which is incorporated by reference in its entirety.

This application is related to U.S. Provisional Application Ser. No. 62/212,173 (Docket No. GTY-002-PR1), titled “Imaging System Includes Imaging Probe and Delivery Devices”, filed Aug. 31, 2015, the content of which is incorporated by reference in its entirety.

This application is related to U.S. Provisional Application Ser. No. 62/368,387 (Docket No. GTY-002-PR2), titled “Imaging System Includes Imaging Probe and Delivery Devices”, filed Jul. 29, 2016, the content of which is incorporated by reference in its entirety.

This application is related to International PCT Patent Application Serial Number PCT/US2016/049415 (Docket No. GTY-002-PCT), titled “Imaging System Includes Imaging Probe and Delivery Devices”, filed Aug. 30, 2016, Publication Number WO 2017/040484, published Mar. 9, 2017, the content of which is incorporated by reference in its entirety.

This application is related to U.S. patent application Ser. No. 15/751,570 (Docket No. GTY-002-US), titled “Imaging System Includes Imaging Probe and Delivery Devices”, filed Feb. 9, 2018, U.S. Pat. No. 10,631,718 , issued Apr. 28, 2020, the content of which is incorporated by reference in its entirety.

This application is related to U.S. patent application Ser. No. 16/820,991 (Docket No. GTY-002-US-CON1), titled “Imaging System Includes Imaging Probe and Delivery Devices”, filed Mar. 17, 2020, U.S. Pat. No. 11,064,873 , issued Jul. 20, 2021, the content of which is incorporated by reference in its entirety.

This application is related to U.S. patent application Ser. No. 17/350,021 (Docket No. GTY-002-US-CON2), titled “Imaging System Includes Imaging Probe and Delivery Devices”, filed Jun. 17, 2021, Publication Number 2022-0142464, published May 12, 2022, the content of which is incorporated by reference in its entirety.

This application is related to U.S. patent application Ser. No. 18/096,678 (Docket No. GTY-002-US-CON3), titled “Imaging System Includes Imaging Probe and Delivery Devices”, filed Jan. 13, 2023, Publication Number published, the content of which is incorporated by reference in its entirety.

This application is related to U.S. Provisional Application Ser. No. 62/591,403 (Docket No. GTY-003-PR1), titled “Imaging System”, filed Nov. 28, 2017, the content of which is incorporated by reference in its entirety.

This application is related to U.S. Provisional Application Ser. No. 62/671,142 (Docket No. GTY-003-PR2), titled “Imaging System”, filed May 14, 2018, the content of which is incorporated by reference in its entirety.

This application is related to International PCT Patent Application Serial Number PCT/US2018/062766 (Docket No. GTY-003-PCT), titled “Imaging System”, filed Nov. 28, 2018, Publication Number WO 2019/108598, published Jun. 6, 2019, the content of which is incorporated by reference in its entirety.

This application is related to U.S. patent application Ser. No. 16/764,087 (Docket No. GTY-003-US), titled “Imaging System”, filed May 14, 2020, Publication Number 2020-0288950, published Sep. 17, 2020, the content of which is incorporated by reference in its entirety.

This application is related to U.S. patent application Ser. No. 18/144,462 (Docket No. GTY-003-US-CON), titled “Imaging System”, filed May 8, 2023, Publication Number, published, the content of which is incorporated by reference in its entirety.

This application is related to U.S. Provisional Application Ser. No. 62/732,114 (Docket No. GTY-004-PR1), titled “Imaging System with Optical Pathway”, filed Sep. 17, 2018, the content of which is incorporated by reference in its entirety.

This application is related to International PCT Patent Application Serial Number PCT/US2019/051447 (Docket No. GTY-004-PCT), titled “Imaging System with Optical Pathway”, filed Sep. 17, 2019, Publication Number WO 2020/061001, published Mar. 26, 2020, the content of which is incorporated by reference in its entirety.

This application is related to U.S. patent application Ser. No. 17/276,500 (Docket No. GTY-004-US), filed Mar. 16, 2021, titled “Imaging system with Optical Pathway”, Publication Number 2021-0267442, published Sep. 2, 2021, the content of which is incorporated by reference in its entirety.

This application is related to U.S. Provisional Application Ser. No. 63/017,258 (Docket No. GTY-005-PR1), titled “Imaging System”, filed Apr. 29, 2020, the content of which is incorporated by reference in its entirety.

This application is related to International PCT Patent Application Serial Number PCT/US2021/29836 (Docket No. GTY-005-PCT), titled “Imaging System”, filed Apr. 29, 2021, Publication Number WO 2021/222530, published Nov. 4, 2021, the content of which is incorporated by reference in its entirety.

This application is related to U.S. patent application Ser. No. 17/919,809 (Docket No. GTY-005-US), filed Oct. 19, 2022, titled “Imaging System”, Publication Number 2023-0181016 published Jun. 15, 2023, the content of which is incorporated by reference in its entirety.

This application is related to U.S. Provisional Application Ser. No. 62/840,450 (Docket No. GTY-011-PR1), titled “Imaging Probe with Fluid Pressurization Element”, filed Apr. 30, 2019, the content of which is incorporated by reference in its entirety.

This application is related to International PCT Patent Application Serial Number PCT/US2020/030616 (Docket No. GTY-011-PCT), titled “Imaging Probe with Fluid Pressurization Element”, filed Apr. 30, 2020, Publication Number WO 2020/223433, published Nov. 5, 2020, the content of which is incorporated by reference in its entirety.

This application is related to U.S. patent application Ser. No. 17/600,212 (Docket No. GTY-011-US), titled “Imaging Probe with Fluid Pressurization Element”, filed Sep. 30, 2021, Publication Number 2022-0142462, published May 12, 2022, the content of which is incorporated by reference in its entirety.

This application is related to U.S. Provisional Application Ser. No. 62/850,945 (Docket No. GTY-013-PR1), titled “OCT-Guided Treatment of a Patient”, filed May 21, 2019, the content of which is incorporated by reference in its entirety.

This application is related to U.S. Provisional Application Ser. No. 62/906,353(GTY-013 -PR2), titled “OCT-Guided Treatment of a Patient”, filed Sep. 26, 2019, the content of which is incorporated by reference in its entirety.

This application is related to International PCT Patent Application Serial Number PCT/US2020/033953 (Docket No. GTY-013-PCT), titled “Systems and Methods for OCT-Guided Treatment of a Patient”, filed May 21, 2020, Publication Number WO 2020/237024, published Nov. 26, 2020, the content of which is incorporated by reference in its entirety.

This application is related to U.S. patent application Ser. No. 17/603,689 (Docket No. GTY-013-US), titled “Systems and Methods for OCT-Guided Treatment of a Patient”, filed Oct. 14, 2021, Publication Number 2022-0061670, published Mar. 3, 2022, the content of which is incorporated by reference in its entirety.

This application is related to U.S. Provisional Application Ser. No. 63/154,934 (Docket No. GTY-021-PR1), titled “Optical Imaging System”, filed Mar. 1, 2021, the content of which is incorporated by reference in its entirety.

This application is related to U.S. patent application Ser. No. 17/682,197 (Docket No. GTY-021-US), titled “Optical Imaging System”, filed Feb. 28, 2022, Publication Number 2023-0000321, published Jan. 5, 2023, the content of which is incorporated by reference in its entirety.

This application is related to U.S. Provisional Application Ser. No. 63/298,086 (Docket No. GTY-022-PR1), titled “Imaging System for Calculating Fluid Dynamics”, filed Jan. 10, 2022, the content of which is incorporated by reference in its entirety.

This application is related to International PCT Patent Application Serial Number PCT/US2023/010508 (Docket No. GTY-022-PCT), titled “Imaging System for Calculating Fluid Dynamics”, filed Jan. 10, 2023, Publication Number WO 2023/133355, published Jul. 13, 2023, the content of which is incorporated by reference in its entirety.

### TECHNICAL FIELD

The present invention relates generally to imaging systems, and in particular, intravascular imaging systems including imaging probes and delivery devices.

### BACKGROUND

Imaging probes have been commercialized for imaging various internal locations of a patient, such as an intravascular probe for imaging a patient's heart. Current imaging probes are limited in their ability to reach certain anatomical locations due to their size and rigidity. Current imaging probes are inserted over a guidewire, which can compromise their placement and limit use of one or more delivery catheters through which the imaging probe is inserted. There is a need for imaging systems that include probes with reduced diameter and high flexibility, as well as systems with one or more delivery devices compatible with these improved imaging probes.

### BRIEF SUMMARY

According to an aspect of the present inventive concepts, an imaging system for a patient comprises an imaging probe comprising: a first elongate shaft comprising a proximal end, a distal portion, a distal end, and a lumen extending at least between the proximal end and the distal portion; a second elongate shaft comprising a proximal end, a distal end, and a lumen extending between the proximal end and the distal end, and at least a portion of the second elongate shaft is positioned within the lumen of the first elongate shaft; a rotatable optical core comprising a proximal end and a distal end, and at least a portion of the rotatable optical core is positioned within the lumen of the second elongate shaft; and an optical assembly positioned proximate the distal end of the rotatable optical core. The optical assembly is configured to direct light to tissue to be imaged and to collect reflected light from the tissue to be imaged. The system further comprises an imaging assembly constructed and arranged to optically couple to the imaging probe, and the imaging assembly is configured to emit light into the imaging probe and to receive the reflected light collected by the optical assembly.

In some embodiments, the distal portion of the first elongate shaft is transparent to the light emitted by the imaging assembly. The second elongate shaft can be not transparent to the light emitted by the imaging assembly.

In some embodiments, the system further comprises a probe interface unit configured to operably attach to the imaging probe. The probe interface unit can be configured to retract the second elongate shaft and the rotatable optical core. The probe interface unit can be configured to rotate the rotatable optical core without rotating either of the first elongate shaft and the second elongate shaft. The imaging probe can further comprise a connector assembly configured to operably connect at least the rotatable optical core and the second elongate shaft to the probe interface unit. The rotatable optical core can further comprise an optical connector, and the connector assembly can comprise a stabilizing assembly configured to engage the optical connector to prevent relative motion between the second elongate shaft and the optical connector. The motion can comprise relative rotational motion. The motion can comprise relative axial motion. The stabilizing assembly can be configured to release the optical connector when the connector assembly is attached to the probe interface unit. The stabilizing assembly can be resiliently biased toward the optical connector to prevent motion when in a resting position. The stabilizing assembly can be resiliently biased away from the optical assembly to allow motion when in a resting position. The connector assembly can further comprise an outer shell configured to bias the stabilizing assembly towards the optical connector.

In some embodiments, the optical assembly is positioned distal to the distal end of the second elongate shaft and within the lumen of the first elongate shaft. The system can further comprise a viscous dampening material positioned within a distal portion of the second elongate shaft and surrounding the rotatable optical core. The imaging probe can be configured such that the viscous dampening material does not exit the second elongate shaft into the first elongate shaft.

In some embodiments, the rotatable optical core extends beyond the distal end of the second elongate shaft and the optical assembly is positioned within the lumen of the first elongate shaft. The outer diameter of the optical assembly can be greater than the inner diameter of the second elongate shaft. The optical assembly can be positioned in air within the lumen of the first elongate shaft. The lumen of the first elongate shaft can be filled with air proximal to and distal to the optical assembly.

In some embodiments, the optical assembly comprises a reflector positioned distal to the distal end of the rotatable optical core. The optical assembly can further comprise a housing, and the housing can be fixedly attached to the reflector and to the distal end of the rotatable optical core. The rotatable optical core and the optical assembly can rotate in unison. The optical assembly can further comprise a space between the reflector and the distal end of the rotatable optical core, and the space can be filled with air. The light exiting the distal end of the rotatable optical core can expand within the space. The reflector can comprise a concave reflecting surface configured to collimate the expanding light.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of a diagnostic system comprising an imaging probe and one or more algorithms for processing image data, consistent with the present inventive concepts.

FIGS. 2, 2A and 2B illustrate a perspective view of an imaging probe, and two sectional views of a portion of the imaging probe, consistent with the present inventive concepts.

FIGS. 3 and 3A illustrate a perspective view and a sectional view of an optical assembly, consistent with the present inventive concepts.

FIGS. 4A-C illustrate perspective views of a reflector, an assembly tool, and an optical assembly, consistent with the present inventive concepts.

FIG. 5 illustrates a perspective view of an assembly tool, consistent with the present inventive concepts.

FIG. 6 illustrates various views of an embodiment of a reflector, consistent with the present inventive concepts.

FIG. 6A-B illustrate an end view and a side view, respectively, of a portion of a reflector, consistent with the present inventive concepts.

FIG. 7 illustrates a side view of the distal end of an optical fiber, consistent with the present inventive concepts.

FIG. 8 illustrates a partially transparent, perspective view of the distal portion of an imaging probe, consistent with the present inventive concepts.

FIGS. 9A and 9B illustrate side sectional and top sectional views, respectively, of the distal end of an imaging probe, consistent with the present inventive concepts.

FIGS. 10A and 10B illustrate optical modeling images and charts of results, consistent with the present inventive concepts.

FIGS. 11A-E illustrate anatomic sectional side views of the steps of an imaging method, consistent with the present inventive concepts.

FIG. 12 illustrates a side view of a portion of an imaging probe, consistent with the present inventive concepts.

FIG. 12A illustrates a side view of a portion of an imaging probe, consistent with the present inventive concepts.

FIG. 13 illustrates a sectional view of the distal end of an imaging probe, consistent with the present inventive concepts.

FIGS. 14, 14A, and 14B illustrate a side view of a lens assembly, the distal end of an optical fiber, and the lens assembly positioned on the distal end of the optical fiber, respectively, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the terms “about” or “approximately” shall refer to ±5% of a stated value.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor, a transducer, or both. In some embodiments, a functional element is configured to deliver energy, delivery a therapeutic treatment, and/or otherwise perform a function. Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems for diagnosing and/or treating a patient, such as to be used in a medical procedure comprising a diagnostic procedure, a therapeutic procedure (also referred to as a “treatment procedure”), or both. The systems of the present inventive concepts comprise an imaging probe and an imaging assembly. The imaging probe can comprise an elongate shaft, a rotatable optical core, and an optical assembly. The shaft can comprise a proximal end, a distal portion, and a lumen extending between the proximal end and the distal portion. The rotatable optical core can comprise a proximal end and a distal end, and at least a portion of the rotatable optical core can be positioned within the lumen of the elongate shaft. The optical assembly can be positioned proximate the distal end of the rotatable optical core and can be configured to direct light to tissue and collect reflected light from the tissue. The imaging systems can comprise one or more algorithms configured to enhance the performance of the system.

The imaging systems of the present inventive concepts can be used to provide image data representing arteries, veins, and/or other body conduits, and to image one or more devices inserted into those conduits. The imaging system can be used to image tissue and/or other structures outside of the blood vessel and/or other lumen into which the imaging probe is inserted. The imaging systems can provide image data related to healthy tissue, as well as diseased tissue, such as blood vessels including a stenosis, myocardial bridge, and/or other vessel narrowing (“lesion” or “stenosis” herein), and/or blood vessels including an aneurysm. The systems can be configured to provide treatment information (e.g. suggested treatment steps to be performed), such as when the treatment information is used by an operator (e.g. a clinician of the patient) to plan a treatment and/or to predict a treatment outcome.

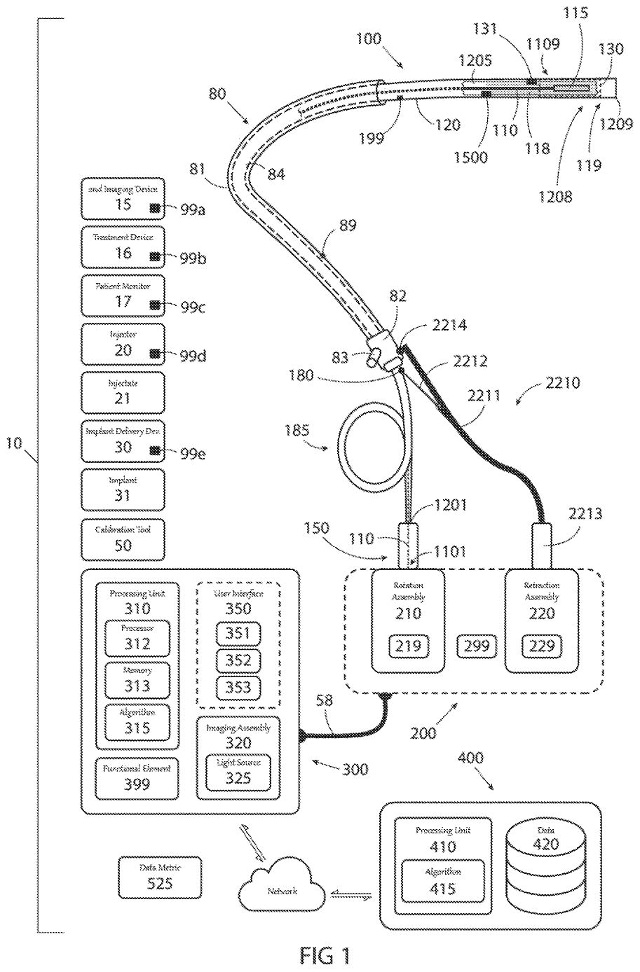

Referring now to FIG. 1, a schematic view of a diagnostic system comprising an imaging probe and one or more algorithms for processing image data is illustrated, consistent with the present inventive concepts. System 10 can be configured as a diagnostic system that is configured to record image data from a patient and produce one or more images based on the recorded data. System 10 can be further configured to analyze the recorded data and/or the produced images (either or both, “image data” herein), such as to provide: diagnostic data relating to a disease or condition of a patient; planning data relating to the planning of a treatment procedure to be performed on a patient; and/or outcome data relating to the efficacy and/or technical outcomes of a treatment procedure.

System 10 can be constructed and arranged to record optical coherence tomography (OCT) data from an imaging location (e.g. OCT data recorded from a segment of a blood vessel during a pullback procedure, as described herein). In some embodiments, the OCT data recorded by system 10 comprises high-frequency OCT (HF-OCT) data. System 10 can comprise a catheter-based probe, imaging probe 100, as well as a probe interface unit, PIU 200, that is configured to operably attach to imaging probe 100. PIU 200 can comprise rotation assembly 210 and/or retraction assembly 220, where each of these can operably attach to imaging probe 100 to rotate and/or retract, respectively, at least a portion of imaging probe 100. System 10 can comprise console 300 that operably attaches to imaging probe 100, such as via PIU 200. Imaging probe 100 can be introduced into a conduit of the patient, such as a blood vessel or other conduit of the patient, using (e.g. passing through) one or more delivery catheters, delivery catheter 80 shown. Additionally or alternatively, imaging probe 100 can be introduced through an introducer device, such as an endoscope, arthroscope, balloon dilator, or the like. In some embodiments, imaging probe 100 is configured to be introduced into a patient conduit and/or other patient internal site selected from the group consisting of: an artery; a vein; an artery within or proximate the heart; a vein within or proximate the heart; an artery within or proximate the brain; a vein within or proximate the brain; a peripheral artery; a peripheral vein; a patient internal site that is accessed through a natural body orifice, such as the esophagus; a patient internal site that is accessed through a surgically created orifice, such as a conduit or other site within the abdomen; and combinations of one or more of these.

In some embodiments, imaging probe 100 and/or another component of system 10 can be of similar construction and arrangement to the similar components described in applicants co-pending U.S. patent application Ser. No. 17/668,757 (Docket No. GTY-001-US-CON1), titled “Micro-Optic Probes for Neurology”, filed Feb. 10, 2022. Imaging probe 100 can be constructed and arranged to collect image data from a patient site, such as an intravascular cardiac site, an intracranial site, or other site accessible via the vasculature of the patient. In some embodiments, system 10 can be of similar construction and arrangement to the similar systems and their methods of use described in applicants co-pending U.S. patent application Ser. No. 18/096,678 (Docket No. GTY-002-US-CON3), titled “Imaging System Includes Imaging Probe and Delivery Devices”, filed Jan. 13, 2023.

Imaging probe 100 can comprise an elongate body comprising one or more elongate shafts and/or tubes, shaft 120 herein. Shaft 120 comprises a proximal end 1201, distal end 1209, and a lumen 1205 extending therebetween. In some embodiments, lumen 1205 can include multiple coaxial lumens within the one or more elongate shafts of shaft 120, such as one or more lumens (e.g. axially aligned lumens) abutting each other to define a single lumen 1205. In some embodiments, at least a portion of shaft 120 comprises a torque shaft. In some embodiments, a portion of shaft 120 comprises a braided construction. In some embodiments, a portion of shaft 120 comprises a spiral cut tube (e.g. shaft 120 includes a spiral cut metal tube). In some embodiments, the pitch of the spiral cut can be varied along the length of the cut, such as to vary the stiffness of shaft 120 along its length. A portion of shaft 120 can comprise a tube constructed of nickel-titanium alloy. Shaft 120 operably surrounds a rotatable optical fiber, optical core 110 (e.g. optical core 110 is positioned within lumen 1205), where core 110 comprises a proximal end 1101 and a distal end 1109. Optical core 110 can comprise a dispersion shifted optical fiber, such as a depressed cladding dispersion shifted fiber (e.g. a Non-Zero Dispersion Shifted, NZDS, fiber). Shaft 120 further comprises a distal portion 1208, including a transparent portion, window 130 (e.g. a window that is relatively transparent to the one or more frequencies of light transmitted through optical core 110). An optical assembly, optical assembly 115, is operably attached to the distal end 1109 of optical core 110. Optical assembly 115 is positioned within window 130 of shaft 120. Optical assembly 115 can comprise a GRIN lens optically coupled to the distal end 1109 of optical core 110. Optical assembly 115 can comprise a construction and arrangement similar to optical assembly 115 as described in applicant's co-pending U.S. patent application Ser. No. 18/144,462 (Docket No. GTY-003-US-CON1), titled “Imaging System”, filed May 8, 2023, and applicant's co-pending U.S. patent application Ser. No. 17/276,500 (Docket No. GTY-004-US), titled “Imaging System with Optical Pathway”, filed Mar. 16, 2021. In some embodiments, optical core 110 comprises a single continuous length of optical fiber comprising zero splices along its length. In some embodiments, imaging probe 100 comprises a single optical splice, such as a splice being between optical assembly 115 and distal end 1109 of optical core 110 (e.g. when there are zero splices along the length of optical core 110).

A connector assembly, connector assembly 150, is positioned on the proximal end of shaft 120. Connector assembly 150 operably attaches imaging probe 100 to rotation assembly 210. In some embodiments, connector assembly 150 comprises an optical connector fixedly attached to the proximal end of optical core 110. Imaging probe 100 can comprise a second connector, connector 180, that can be positioned on shaft 120. Connector 180 can be removably attached and/or adjustably positioned along the length of shaft 120. Connector 180 can be positioned along shaft 120, such as by a clinician, technician, and/or other user of system 10 (“user” or “operator” herein), proximate the proximal end of delivery catheter 80 after imaging probe 100 has been inserted into a patient via delivery catheter 80. Shaft 120 can comprise a portion between connector assembly 150 and the placement location of connector 180 that is configured to provide and/or accommodate slack in shaft 120, service loop 185.

In some embodiments, shaft 120 comprises a multi-part construction, such as an assembly of two or more tubes that can be connected in various ways. In some embodiments, one or more tubes of shaft 120 can comprise tubes made of polyethylene terephthalate (PET), such as when a PET tube surrounds the junction between two tubes (e.g. two portions of shaft 120) in an axial arrangement to create a joint between the two tubes. In some embodiments, one or more PET tubes are under tension after assembly (e.g. the tubes are longitudinally stretched when shaft 120 is assembled), such as to prevent or at least reduce the tendency of the PET tube to wrinkle while shaft 120 is advanced through a tortuous path. In some embodiments, one or more portions of shaft 120 include a coating comprising one, two, or more materials and/or surface modifying processes, such as to provide a hydrophilic coating or a lubricious coating. In some embodiments, one or more metal portions of shaft 120 (e.g. nickel-titanium portions) are surrounded by a tube (e.g. a polymer tube), such as to improve the adhesion of a coating to that portion of shaft 120.

Imaging probe 100 can comprise one or more visualizable markers along its length (e.g. along shaft 120), marker 131 shown. Marker 131 can comprise one or more markers selected from the group consisting of: radiopaque markers; ultrasonically reflective markers; magnetic markers; ferrous material; and combinations of one or more of these. In some embodiments, marker 131 is positioned at a location along imaging probe 100 selected to assist an operator of system 10 in performing a pullback procedure (“pullback procedure” or “pullback” herein). For example, marker 131 can be positioned approximately one pullback length from distal end 1209 of shaft 120, such that following a pullback, distal end 1209 will be no more proximal than the starting position of marker 131. In some embodiments, prior to a pullback, the operator can position marker 131 at a location distal to the proximal end of an implant, such that after the pullback is completed access into the implant is maintained (e.g. such that imaging probe 100 can be safely advanced through the implant after the pullback).

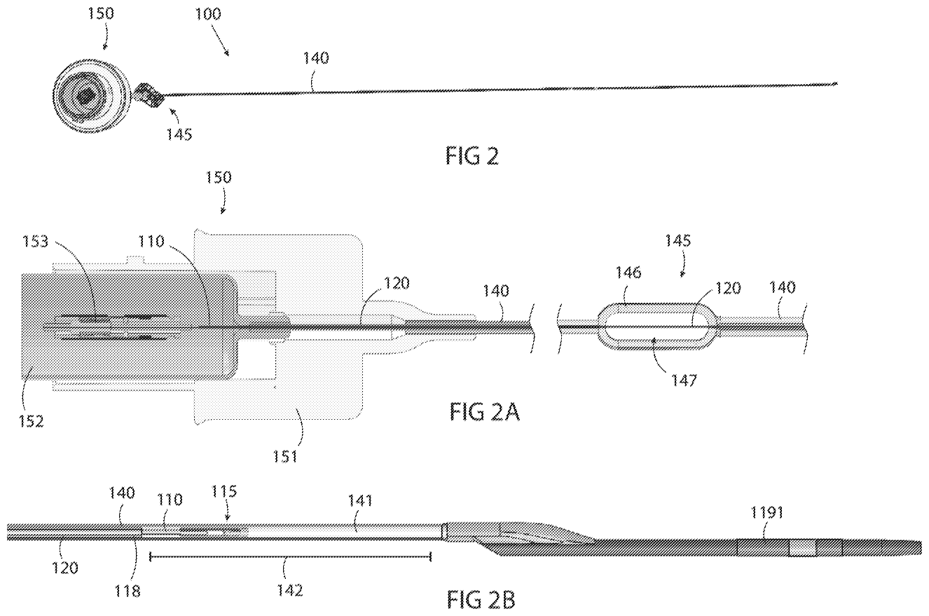

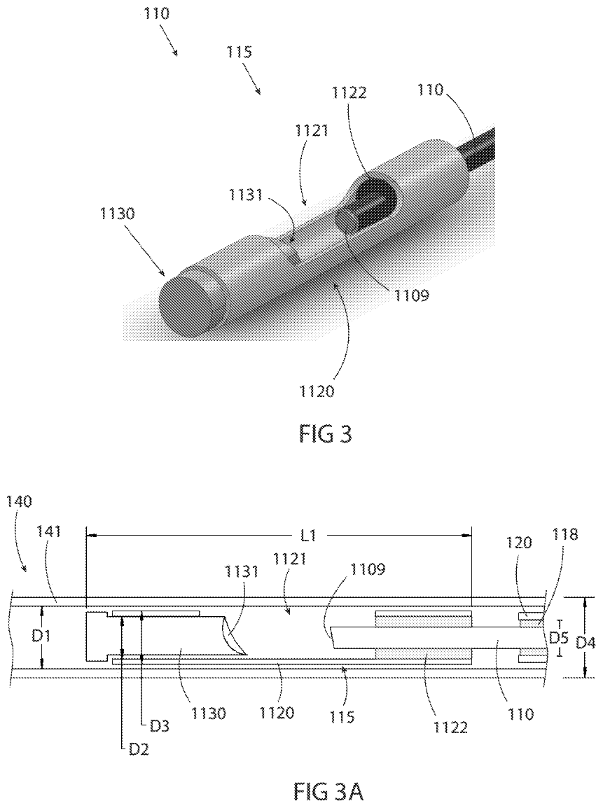

In some embodiments, imaging probe 100 includes a viscous dampening material, gel 118. Gel 118 can be positioned within shaft 120 surrounding a distal portion of optical core 110 (e.g. a gel injected or otherwise installed in a manufacturing process). In some embodiment, gel 118 surrounds a portion of optical assembly 115. Alternatively, gel 118 does not surround a portion of optical assembly 115, for example when optical assembly 115 is configured to operate in air, such as is described hereinbelow in reference to FIGS. 2 and 3. Gel 118 can comprise a non-Newtonian fluid, for example a shear-thinning fluid. In some embodiments, gel 118 comprises a static viscosity of at least 500 centipoise, and a shear viscosity that is less than the static viscosity. In these embodiments, the ratio of static viscosity to shear viscosity of gel 118 can be between 1.2:1 and 100:1. In some embodiments, gel 118 is injected from the distal end of window 130 (e.g. in a manufacturing process). In some embodiments, gel 118 comprises a gel which is visualizable (e.g. visualizable under UV light, such as when gel 118 includes one or more materials that fluoresce under UV light). In some embodiments, during a manufacturing process in which gel 118 is injected into shaft 120 via window 130, shaft 120 is monitored while gel 118 is visualized (e.g. being illuminated by UV light) such that the injection process can be controlled (e.g. injection is stopped when gel 118 sufficiently ingresses into shaft 120). Gel 118 can comprise a gel as described in reference to applicants co-pending U.S. patent application Ser. No. 17/668,757 (Docket No. GTY-001-US-CON1), titled “Micro-Optic Probes for Neurology”, filed Feb. 10, 2022, and applicant's co-pending U.S. patent application Ser. No. 18/144,462 (Docket No. GTY-003-US-CON1), titled “Imaging System”, filed May 8, 2023.

Imaging probe 100 can include a distal tip portion, distal tip 119. In some embodiments, distal tip 119 can comprise a spring tip, such as a spring tip configured to improve the “navigability” of imaging probe 100 (e.g. to improve “trackability” and/or “steerability” of imaging probe 100), for example when probe 100 is translated within a tortuous pathway (e.g. within a blood vessel of the brain or heart with a tortuous pathway). In some embodiments, distal tip 119 comprises a length of between 5 mm and 100 mm (e.g. a spring with a length between 5 mm and 100 mm). In some embodiments, distal tip 119 can comprise a user shapeable spring tip (e.g. at least a portion of distal tip 119 is malleable). Imaging probe 100 can be rotated (e.g. via connector 180) to adjust the direction of a non-linear shaped portion of distal tip 119 (e.g. to adjust the trajectory of distal tip 119 in the vasculature of the patient). Alternatively or additionally, distal tip 119 can comprise a cap, plug, and/or other element configured to seal the distal opening of window 130. In some embodiments, distal tip 119 can comprise a radiopaque marker configured to increase the visibility of imaging probe 100 under a fluoroscope or other X-ray device. In some embodiments, distal tip 119 can comprise a relatively short luminal guidewire pathway to allow “rapid exchange” translation of imaging probe 100 over a guidewire of system 10 (guidewire not shown).

In some embodiments, at least the distal portion of imaging probe 100 (e.g. the distal portion of shaft 120 surrounding optical assembly 115) comprises an outer diameter of no more than 0.030″, such as no more than 0.025″, no more than 0.020″, and/or no more than 0.016″.

In some embodiments, imaging probe 100 can be constructed and arranged for use in an intravascular neural procedure (e.g. a procedure in which the blood, vasculature, and/or other tissue proximate the brain are visualized, and/or devices positioned temporarily or permanently proximate the brain are visualized). An imaging probe 100 configured for use in an intravascular neural procedure (also referred to herein as a “neural procedure”) can comprise an overall length of at least 150 cm, such as a length of approximately 300 cm. Alternatively or additionally, imaging probe 100 can be constructed and arranged for use in an intravascular cardiac procedure (e.g. a procedure in which the blood, vasculature, and other tissue proximate the heart are visualized, and/or devices positioned temporarily or permanently proximate the heart are visualized). An imaging probe 100 configured for use in an intravascular cardiac procedure (e.g. also referred to as a “cardiac procedure” or “cardiovascular procedure” herein) can comprise an overall length of at least 120 cm, such as an overall length of approximately 280 cm (e.g. to allow placement of the proximal end of imaging probe 100 outside of the sterile field). In some embodiments, such as for placement of the proximal end of probe 100 outside of the sterile field, imaging probe 100 can comprise a length greater than 220 cm, such as a length of at least 220 cm but less than 320 cm.

In some embodiments, imaging probe 100 comprises an element, FPE 1500 shown, which can be configured as a fluid propulsion element and/or a fluid pressurization element (“fluid pressurization element” herein). FPE 1500 can be configured to prevent and/or reduce the presence of bubbles within gel 118 proximate optical assembly 115. FPE 1500 can be fixedly attached to optical core 110, wherein rotation of optical core 110 in turn rotates FPE 1500, such as to generate a pressure increase within gel 118 that is configured to reduce presences of bubbles from locations proximate optical assembly 115. Such one or more fluid pressurization elements FPE 1500 can be constructed and arranged to: reduce the likelihood of bubble formation within gel 118, reduce the size of bubbles within gel 118, and/or move any bubbles formed within gel 118 away from a location that would adversely impact the collecting of image data by optical assembly 115 (e.g. move bubbles away from optical assembly 115). In some embodiments, a fluid propulsion element FPE 1500 of imaging probe 100 comprises a similar construction and arrangement to a fluid propulsion element described in applicant's co-pending U.S. patent application Ser. No. 17/600,212 (Docket No. GTY-011-US), titled “Imaging Probe with Fluid Pressurization Element”, filed Sep. 30, 2021.

In some embodiments, delivery catheter 80 comprises an elongate shaft, shaft 81 shown, which includes a lumen 84 therethrough and a connector 82 positioned on its proximal end. Connector 82 can comprise a Touhy or other valved connector, such as a valved connector configured to prevent fluid egress from the associated delivery catheter 80 (with and/or without a separate shaft positioned within the connector 82). Connector 82 can comprise port 83, such as one or more ports constructed and arranged to allow introduction of fluid into delivery catheter 80 and/or for removing fluids from delivery catheter 80. In some embodiments, a flushing fluid, such as is described herein, is introduced via one or more ports 83, such as to remove blood or other undesired material from locations proximate optical assembly 115 (e.g. from a location proximal to optical assembly 115 to a location distal to optical assembly 115). Port 83 can be positioned on a side of connector 82 and can include a luer fitting and a cap and/or valve. Shafts 81, connectors 82, and ports 83 can each comprise standard materials and be of similar construction to commercially available introducers, guide catheters, diagnostic catheters, intermediate catheters and microcatheters used in interventional procedures today. Delivery catheter 80 can comprise a catheter configured to deliver imaging probe 100 to an intracerebral location, an intracardiac location, and/or another location within a patient.

Delivery catheter 80 can comprise two or more delivery catheters, such as three or more delivery catheters. Delivery catheter 80 can comprise at least a vascular introducer, and other delivery catheters that can be inserted into the patient (e.g. through the vascular introducer, after the vascular introducer is positioned through the skin of the patient). Delivery catheter 80 can comprise sets of two or more delivery catheters collectively comprising sets of various inner diameters (IDs) and outer diameters (ODs) such that a first delivery catheter 80 slidingly receives a second delivery catheter 80 (e.g. the second delivery catheter OD is less than or equal to the first delivery catheter ID), and the second delivery catheter 80 slidingly receives a third delivery catheter 80 (e.g. the third delivery catheter OD is less than or equal to the second delivery catheter ID), and so on. In these configurations, the first delivery catheter 80 (e.g. its distal end) can be advanced to a first anatomical location, the second delivery catheter 80 (e.g. its distal end) can be advanced through the first delivery catheter to a second anatomical location distal or otherwise remote (hereinafter “distal”) to the first anatomical location, and so on as appropriate, using sequentially smaller diameter delivery catheters. In some embodiments, delivery catheter 80 can be of similar construction and arrangement to the similar components described in applicants co-pending U.S. patent application Ser. No. 18/096,678 (Docket No. GTY-002-US-CON3), titled “Imaging System Includes Imaging Probe and Delivery Devices”, filed Jan. 13, 2023.

In some embodiments, delivery catheter 80 comprises a guide extension catheter, such as a catheter including a coil-reinforced hollow shaft, and a push wire attached to the proximal end of the shaft. The shaft can include a skived (partial circumferential) proximal portion for ease of insertion of a separate device (e.g. a treatment device and/or probe 100) through the shaft.

Rotation assembly 210 operably attaches to connector assembly 150 of imaging probe 100. Rotation assembly 210 can comprise one or more rotary joints, optical connectors, rotational actuators (e.g. motors), and/or linkages, configured to operably attach to, allow the rotation of, and/or cause the rotation of optical core 110. Connector assembly 150 can be constructed and arranged to removably attach to rotation assembly 210, and to allow a rotating connection between proximal end 1101 and a rotating fiber optic joint (such as a fiber optic rotary joint or FORJ). Rotation assembly 210 can be of similar construction and arrangement to similar components described in applicant's co-pending U.S. patent application Ser. No. 18/144,462 (Docket No. GTY-003-US-CON1), titled “Imaging System”, filed May 8, 2023, and applicant's co-pending U.S. patent application Ser. No. 17/276,500 (Docket No. GTY-004-US), titled “Imaging System with Optical Pathway”, filed Mar. 16, 2021. Rotation assembly 210 can be configured to rotate optical core 110 at speeds of at least 100 rotations per second, such as at least 200 rotations per second or 250 rotations per second, or at speeds between 20 rotations per second and 1000 rotations per second. Rotation assembly 210 can comprise a rotational actuator selected from the group consisting of: a motor; a servo; a stepper motor (e.g. a stepper motor including a gear box); an actuator; a hollow core motor; and combinations thereof. In some embodiments, rotation assembly 210 is configured to rotate optical assembly 115 and optical core 110 in unison.

Retraction assembly 220 operably attaches to imaging probe 100, such as to retract imaging probe 100 relative to a patient access site. A retraction element 2210 can operably attach to retraction assembly 220 and imaging probe 100, such as to transfer a retraction force from retraction assembly 220 to imaging probe 100. Retraction element 2210 can comprise a conduit 2211, surrounding a linkage 2212, slidingly received therein. Retraction element 2210 can comprise a connector 2213 that operably attaches to retraction assembly 220, such that retraction assembly 220 can retract linkage 2212 relative to conduit 2211. In some embodiments, conduit 2211 comprises a connector 2214 that operably attaches to a reference point near the patient access site, for example to connector 82 of delivery catheter 80, such as to establish a reference for retraction of imaging probe 100 relative to the patient. Connector 2214 can attach to a reference point such as by attaching to a patient introduction device, surgical table, and/or another fixed or semi fixed point of reference. Linkage 2212 releasably attaches to connector 180 of imaging probe 100. Retraction assembly 220 retracts at least a portion of imaging probe 100 (e.g. the portion of imaging probe 100 distal to the attached connector 180) relative to the established reference by retracting linkage 2212 relative to conduit 2211 (e.g. retract a portion of linkage 2212 exiting a portion of conduit 2211, as shown). In some embodiments, retraction assembly 220 is configured to retract at least a portion of imaging probe 100 (e.g. at least optical assembly 115 and a portion of shaft 120) at a rate of between 5 mm/sec and 200 mm/sec, or between 5 mm/sec and 100 mm/sec, such as a rate of approximately 60 mm/sec. Additionally or alternatively, a pullback procedure can be performed during a time period of between 0.5 sec and 25 sec, for example approximately 20 sec (e.g. over a distance of 100 mm at 5 mm/sec). Service loop 185 of imaging probe 100 can be positioned between connector 180, and rotation assembly 210, such that imaging probe 100 can be retracted relative to the patient while rotation assembly 210 remains stationary (e.g. attached to the surgical table and/or to a portion of console 300).

Retraction assembly 220 further comprises a motive element configured to retract linkage 2212. In some embodiments, the motive element comprises a linear actuator, a worm drive operably attached to a motor, a pulley system, and/or other linear force transfer mechanisms. Linkage 2212 can be operably attached to the motive element via one or more linkages and/or connectors. Retraction assembly 220 can be of similar construction and arrangement to similar components described in applicant's co-pending U.S. patent application Ser. No. 18/144,462 (Docket No. GTY-003-US-CON1), titled “Imaging System”, filed May 8, 2023.

In some embodiments, PIU 200 can comprise a single discrete component (e.g. a single housing) which can contain both rotation assembly 210 and retraction assembly 220. Alternatively or additionally, PIU 200 can comprise two or more discrete components (e.g. two or more housings), such as a separate component for each of rotation assembly 210 and retraction assembly 220. In some embodiments, connector assembly 150, service loop 185, retraction element 2210, and connector 2213 are included in a single discrete component (e.g. housed within a single housing) and configured to operably attach to both rotation assembly 210 and retraction assembly 220 (e.g. such as when rotation assembly 210 and retraction assembly 220 are housed within a single housing or otherwise included in a single discrete component).

In some embodiments, system 10 includes a supplementary imaging device (e.g. in addition to imaging probe 100), second imaging device 15. Second imaging device 15 can comprise an imaging device such as one or more imaging devices selected from the group consisting of: an X-ray; a fluoroscope such as a single plane or biplane fluoroscope; a CT Scanner; an MRI; a PET Scanner; an ultrasound imager; and combinations of one or more of these. In some embodiments, second imaging device 15 comprises a device configured to perform rotational angiography.

In some embodiments, system 10 includes a device configured to treat the patient (e.g. provide one or more therapies to the patient), treatment device 16. Treatment device 16 can comprise an occlusion treatment device and/or other treatment device selected from the group consisting of: a balloon catheter constructed and arranged to dilate a stenosis or other narrowing of a blood vessel; a drug eluting balloon; an aspiration catheter; a sonolysis device; an atherectomy device; a thrombus removal device such as a stent retriever device; a Trevo™ stentriever; a Solitaire™ stentriever; a Revive™ stentriever; an Eric™ stentriever; a Lazarus™ stentriever; a stent delivery catheter; a microbraid implant; an embolization system; a WEB™ embolization system; a Luna™ embolization system; a Medina™ embolization system; and combinations of one or more of these. In some embodiments, imaging probe 100 and/or another component of system 10 is configured to collect data related to treatment device 16 (e.g. treatment device 16 location, orientation and/or other configuration data), after treatment device 16 has been inserted into the patient.

System 10 can further comprise one or more devices that are configured to monitor one, two, or more physiologic and/or other parameters of the patient, such as patient monitor 17 shown. Patient monitor 17 can comprise one or more monitoring devices selected from the group consisting of: an ECG monitor; an EEG monitor; a blood pressure monitor; a blood flow monitor; a respiration monitor; a patient movement monitor; a T-wave trigger monitor; and combinations of these.

System 10 can further comprise one or more fluid injectors, injector 20 shown, each of which can be configured to inject one or more fluids, such as a flushing fluid, an imaging contrast agent (e.g. a radiopaque contrast agent, hereinafter “contrast”) and/or other fluid, such as injectate 21 shown. Injector 20 can comprise a power injector, syringe pump, peristaltic pump or other fluid delivery device configured to inject a contrast agent, such as radiopaque contrast, and/or other fluids. In some embodiments, injector 20 is configured to deliver contrast and/or other fluid (e.g. contrast, saline, and/or dextran). In some embodiments, injector 20 delivers fluid in a flushing procedure, such as is described herein. In some embodiments, injector 20 delivers contrast or other fluid through delivery catheter 80 comprising an ID of between 5 Fr and 9 Fr, a delivery catheter 80 comprising an ID of between 0.53″ to 0.70″, or a delivery catheter 80 comprising an ID between 0.0165″ and 0.027″. In some embodiments, contrast or other fluid is delivered through a delivery catheter as small as 4 Fr (e.g. for distal injections). In some embodiments, injector 20 delivers contrast and/or other fluid through the lumen of delivery catheter 80, while one or more smaller delivery catheters 80 also reside within the lumen of delivery catheter 80. In some embodiments, injector 20 is configured to deliver two dissimilar fluids simultaneously and/or sequentially, such as a first fluid delivered from a first reservoir and comprising a first concentration of contrast, and a second fluid from a second reservoir and comprising less or no contrast.

Injectate 21 can comprise fluid selected from the group consisting of: optically transparent material; saline; visualizable material; contrast; dextran; an ultrasonically reflective material; a magnetic material; and combinations thereof. Injectate 21 can comprise contrast and saline. Injectate 21 can comprise at least 20% contrast. During collection of image data (e.g. during a pullback), a flushing procedure can be performed, such as by delivering one or more fluids, (e.g. injectate 21 as propelled by injector 20 or other fluid delivery device), to remove blood or other somewhat opaque material (hereinafter non-transparent material) proximate optical assembly 115 (e.g. to remove non-transparent material between optical assembly 115 and a delivery catheter and/or non-transparent material between optical assembly 115 and a vessel wall), such as to allow light distributed from optical assembly 115 to reach and reflectively return from all tissue and other objects to be imaged. In these flushing embodiments, injectate 21 can comprise an optically transparent material, such as saline. Injectate 21 can comprise one or more visualizable materials, as described herein.

As an alternative or in addition to its use in a flushing procedure, injectate 21 can comprise material configured to be viewed by second imaging device 15, such as when injectate 21 comprises a contrast material configured to be viewed by a second imaging device 15 comprising a fluoroscope and/or other X-ray device; an ultrasonically reflective material configured to be viewed by a second imaging device 15 comprising an ultrasound imager; and/or a magnetic material configured to be viewed by a second imaging device 15 comprising an MRI.

System 10 can further comprise an implant, such as implant 31, which can be implanted in the patient via a delivery device, such as an implant delivery device 30 and/or delivery catheter 80. Implant 31 can comprise an implant (e.g. a temporary or chronic implant) for treating, for example, a vascular occlusion and/or an aneurysm. In some embodiments, implant 31 comprises one or more implants selected from the group consisting of: a flow diverter; a Pipeline™ flow diverter; a Surpass™ flow diverter; an embolization coil; a stent; a Wingspan™ stent; a covered stent; an aneurysm treatment implant; and combinations of one or more of these.

Implant delivery device 30 can comprise a catheter and/or other tool used to deliver implant 31, such as when implant 31 comprises a self-expanding or balloon expandable portion. In some embodiments, system 10 comprises imaging probe 100, one or more implants 31 and/or one or more implant delivery devices 30. In some embodiments, imaging probe 100 is configured to collect data related to implant 31 and/or implant delivery device 30 (e.g. implant 31 and/or implant delivery device 30 anatomical location, orientation and/or other configuration data), after implant 31 and/or implant delivery device 30 has been inserted into the patient.

In some embodiments, one or more system 10 components, such as second imaging device 15, treatment device 16, patient monitor 17, injector 20, implant delivery device 30, delivery catheter 80, imaging probe 100, PIU 200, rotation assembly 210, retraction assembly 220, and/or console 300, further comprise one or more functional elements (“functional element” herein), such as functional elements 99a, 99b, 99c, 99d, 99e, 89, 199, 299, 219, 229, and/or 399, respectively, each as shown. Each functional element can comprise at least two functional elements. Each functional element can comprise one or more elements selected from the group consisting of: sensor; transducer; and combinations thereof. The functional element can comprise a sensor configured to produce a signal. The functional element can comprise a sensor selected from the group consisting of: a physiologic sensor; a pressure sensor; a strain gauge; a position sensor; a GPS sensor; an accelerometer; a temperature sensor; a magnetic sensor; a chemical sensor; a biochemical sensor; a protein sensor; a flow sensor such as an ultrasonic flow sensor; a gas detecting sensor such as an ultrasonic bubble detector; a sound sensor such as an ultrasound sensor; and combinations thereof. The sensor can comprise a physiologic sensor selected from the group consisting of: a pressure sensor such as a blood pressure sensor; a blood gas sensor; a flow sensor such as a blood flow sensor; a temperature sensor such as a blood or other tissue temperature sensor; and combinations thereof. The sensor can comprise a position sensor configured to produce a signal related to a vessel path geometry (e.g. a 2D or 3D vessel path geometry). The sensor can comprise a magnetic sensor. The sensor can comprise a flow sensor. The system can further comprise an algorithm configured to process the signal produced by the sensor-based functional element. Each functional element can comprise one or more transducers. Each functional element can comprise one or more transducers selected from the group consisting of: a heating element such as a heating element configured to deliver sufficient heat to ablate tissue; a cooling element such as a cooling element configured to deliver cryogenic energy to ablate tissue; a sound transducer such as an ultrasound transducer; a vibrational transducer; and combinations thereof.

In some embodiments, imaging probe 100 comprises an overall length of at least 120 cm, such as at least 160 cm, such as approximately 280 cm. In some embodiments, imaging probe 100 comprises an overall length of no more than 350 cm. In some embodiments, imaging probe 100 comprises a length configured to be inserted into the patient (“insertable length” herein) of at least 90 cm, such as at least 100 cm, such as approximately 145 cm. In some embodiments, imaging probe 100 comprises an insertable length of no more than 250 cm, such as no more than 200 cm. In some embodiments, distal tip 119 comprises a spring tip with a length of at least 5 mm, such as at least 25 mm, such as approximately 15 mm. In some embodiments, distal tip 119 comprises a spring tip with a length of no more than 75 mm, such as no more than 30 mm. In some embodiments, a distal portion of shaft 120 (e.g. window 130) comprises an outer diameter of less than 2 Fr, such as less than 1.4 Fr, such as approximately 1.1 Fr. In some embodiments, a distal portion of shaft 120 (e.g. window 130) comprises an outer diameter of at least 0.5 Fr, such as at least 0.9 Fr. In some embodiments, shaft 120 comprises one or more materials selected from the group consisting of: polyether ether ketone (PEEK); nylon; polyether block amide; nickel-titanium alloy; and combinations of these.

In some embodiments, at least a portion of imaging probe 100 (e.g. the most flexible portion) is configured to safely and effectively be positioned in a radius of curvature as low as 5 mm, 4 mm, 3 mm, 2 mm, and/or 1 mm. In some embodiments optical core 110 comprises an optical fiber with a diameter of less than 120 μm, such as less than 100 μm, such as less than 80 μm, such as less than 60 μm, such as approximately 40 μm. In some embodiments, optical core 110 comprises a numerical aperture of one or more of 0.11, 0.14, 0.16, 0.17, 0.18, 0.20, and/or 0.25. In some embodiments, optical assembly 115 comprises a lens selected from the group consisting of: a GRIN lens; a molded lens; a shaped lens, such as a melted and polished lens; a lens comprising an axicon structure, (e.g. an axicon nano-structure); and combinations of these. In some embodiments, optical assembly 115 comprises a lens with an outer diameter of less than 200 μm, such as less than 170 μm, such as less than 150 μm, such as less than 100 μm, such as approximately 80 μm. In some embodiments optical assembly 115 comprises a lens with a length of less than 3 mm, such as less than 1.5 mm. In some embodiments, optical assembly 115 comprises a lens with a length of at least 0.5 mm, such as at least 1 mm. In some embodiments, optical assembly 115 comprises a lens with a focal length of at least 0.5 mm and/or no more than 5.0 mm, such as at least 1.0 mm and/or no more than 3.0 mm, such as a focal length of approximately 0.5 mm. In some embodiments, optical assembly 115 can comprise longer focal lengths, such as to view structures outside of the blood vessel in which optical assembly 115 is inserted. In some embodiments, optical assembly 115 has a working distance (also termed depth of field, confocal distance, or Rayleigh Range) of up to 1 mm, such as up to 5 mm, such as up to 10 mm, such as a working distance of at least 1 mm and/or no more than 5 mm. In some embodiments, optical assembly 115 comprises an outer diameter of at least 80 μm and/or no more than 200 μm, such as at least 150 μm and/or no more than 170 μm, such as an outer diameter of approximately 150 μm. In some embodiments, system 10 (e.g. retraction assembly 220) is configured to perform a pullback of imaging probe 100 at a speed of at least 10 mm/sec and/or no more than 300 mm/sec, such as at least 50 mm/sec and/or no more than 200 mm/sec, such as a pullback speed of approximately 100mm/sec. In some embodiments, system 10 (e.g. retraction assembly 220) is configured to perform a pullback for a distance of at least 25 mm and/or no more than 200 mm, such as at least 25 mm and/or no more than 150 mm, such as a distance of approximately 50 mm. In some embodiments, system 10 (e.g. retraction assembly 220) is configured to perform a pullback over a time period of at least 0.2 seconds and/or no more than 5.0 seconds, such as at least 0.5 seconds and/or no more than 2.0 seconds, such as a time period of approximately 1.0 second. In some embodiments, system 10 (e.g. rotation assembly 210) is configured to rotate optical core 110 at an angular velocity of at least 20 rotations per second and/or no more than 1000 rotations per second, such as at least 100 rotations per second and/or no more than 500 rotations per second, such as an angular velocity of approximately 250 rotations per second. In some embodiments, delivery catheter 80 comprises an inner diameter of at least 0.016″ and/or no more than 0.050″, such as at least 0.016″ and/or no more than 0.027″, such as an inner diameter of approximately 0.021″.

In some embodiments, console 300 comprises imaging assembly 320 that can be configured to provide light to optical assembly 115 (e.g. via optical core 110) and collect light from optical assembly 115 (e.g. via optical core 110). Imaging assembly 320 can include a light source 325. Light source 325 can comprise one or more light sources, such as one or more light sources configured to provide one or more wavelengths of light to optical assembly 115 via optical core 110. Light source 325 is configured to provide light to optical assembly 115 (via optical core 110) such that image data can be collected comprising cross-sectional, longitudinal and/or volumetric information related to a patient site or implanted device being imaged. Light source 325 can be configured to provide light such that the image data collected includes characteristics of tissue within the patient site being imaged, such as to quantify, qualify or otherwise provide information related to a patient disease or disorder present within the patient site being imaged. Light source 325 can be configured to deliver broadband light and have a center wavelength in the range from 350 nm to 2500 nm, from 800 nm to 1700 nm, from 1280 nm to 1310 nm, or approximately 1300 nm (e.g. light delivered with a sweep range from 1250nm to 1350nm). Light source 325 can comprise a sweep rate of at least 20 kHz. In some embodiments, light source 325 comprises a sweep rate of at least 100 KHz, such as at least 200 Khz, 300 KHz, 400 KHz, and/or 500 KHz, for example approximately 200 kHz. These faster sweep rates provide numerous advantages (over similar systems comprising slower sweep rates), such as to provide a higher frame rate, as well as being compatible with rapid pullback and rotation rates. For example, the higher sweep rate enables the requisite sampling density (e.g. the amount of luminal surface area swept by the rotating beam) to be achieved in a shorter time, advantageous in most situations and especially advantageous when there is relative motion between the probe and the surface/tissue being imaged such as arteries in a beating heart. Light source 325 bandwidth can be selected to achieve a desired resolution, which can vary according to the needs of the intended use of system 10. In some embodiments, bandwidths are about 5% to 15% of the center wavelength, which allows resolutions of between 20 μm and 5 μm. Light source 325 can be configured to deliver light at a power level meeting ANSI Class 1 (“eye safe”) limits, though higher power levels can be employed. In some embodiments, light source 325 delivers light in the 1.3 μm band at a power level of approximately 20 mW. Tissue light scattering is reduced as the center wavelength of delivered light increases, however water absorption increases. Light source 325 can deliver light at a wavelength approximating 1300 nm to balance these two effects. Light source 325 can be configured to deliver shorter wavelength light (e.g. approximately 800 nm light) to traverse patient sites to be imaged including large amounts of fluid. Alternatively or additionally, light source 325 can be configured to deliver longer wavelengths of light (e.g. approximately 1700 nm light), such as to reduce a high level of scattering within a patient site to be imaged. In some embodiments, light source 325 comprises a tunable light source (e.g. light source 325 emits a single wavelength that changes repetitively over time), and/or a broad-band light source. Light source 325 can comprise a single spatial mode light source or a multimode light source (e.g. a multimode light source with spatial filtering).

Light source 325 can comprise a relatively long effective coherence length, such as a coherence length of greater than 10 mm, such as a length of at least 50 mm, at all frequencies within the bandwidth of the light source. This coherence length capability enables longer effective scan ranges to be achieved by system 10, as the light returning from distant objects to be imaged (e.g. tissue) must remain in phase coherence with the returning reference light, in order to produce detectable interference fringes. In the case of a swept-source laser, the instantaneous linewidth is very narrow (i.e. as the laser is sweeping, it is outputting a very narrow frequency band that changes at the sweep rate). Similarly, in the case of a broad-bandwidth source, the detector arrangement must be able to select very narrow linewidths from the spectrum of the source. The coherence length scales inversely with the linewidth. Longer scan ranges enable larger or more distant objects to be imaged (e.g. more distal tissue to be imaged). Current systems have lower coherence length, which correlates to reduced image capture range as well as artifacts (ghosts) that arise from objects outside the effective scan range.

In some embodiments, light source 325 comprises a sweep bandwidth of at least 30 nm and/or no more than 250 nm, such as at least 50 nm and/or no more than 150 nm, such as a sweep bandwidth of approximately 100 nm. In some embodiments, light source 325 comprises a center wavelength of at least 800 nm and/or no more than 1800 nm, such as at least 1200 nm and/or no more than 1350 nm, such as a center wavelength of approximately 1300 nm. In some embodiments, light source 325 comprises an optical power of at least 5 mW and/or no more than 500 mW, such as at least 10 mW and/or no more than 50 mW, such as an optical power of approximately 20 mW.

System 10 can comprise one or more operably-connecting cables or other conduits, bus 58 shown. Bus 58 can operably connect PIU 200 to console 300, rotation assembly 210 to console 300 (as shown), retraction assembly 220 to console 300, and/or rotation assembly 210 to retraction assembly 220. Bus 58 can comprise one or more optical transmission fibers, wires, traces, and/or other electrical transmission cables, fluid conduits, and combinations of one or more of these. In some embodiments, bus 58 comprises at least an optical transmission fiber that optically couples rotation assembly 210 to imaging assembly 320 of console 300. Additionally or alternatively, bus 58 comprises at least power and/or data transmission cables that transfer power and/or drive signals to one or more of motive elements of rotation assembly 210 and/or retraction assembly 220.

Console 300 can include processing unit 310, which can be configured to perform and/or facilitate one or more functions of system 10, such as one or more processes, energy deliveries (e.g. light energy deliveries), data collections, data analyses, data transfers, signal processing, and/or other functions (“functions” herein). Processing unit 310 can include processor 312, memory 313, and/or algorithm 315, each as shown. Memory 313 can store instructions for performing algorithm 315 and can be coupled to processor 312. System 10 can include an interface, user interface 350, for providing and/or receiving information to and/or from an operator of system 10. User interface 350 can be integrated into console 300 as shown. In some embodiments, user interface 350 can comprise a component separate from console 300, such as a display separate from, but operably attached to, console 300. User interface 350 can include one, two, or more user input and/or user output components. For example, user interface 350 can comprise a joystick, keyboard, mouse, touchscreen, and/or another human interface device, user input device 351 shown. In some embodiments, user interface 350 comprises a display (e.g. a touchscreen display), such as display 352, also shown. In some embodiments, processor 312 can provide a graphical user interface, GUI 353, to be presented on and/or provided by display 352. User interface 350 can include an input and/or output device selected from the group consisting of: a speaker; an indicator light, such as an LED indicator; a haptic feedback device; a foot pedal; a switch such as a momentary switch; a microphone; a camera, for example when processor 312 enables eye tracking and/or other input via image processing; and combinations of these.

In some embodiments, system 10 includes a data storage and processing device, server 400. Server 400 can comprise an “off-site” server (e.g. outside of the clinical site in which patient image data is recorded), such as a server owned, maintained, and/or otherwise provided by the manufacturer of system 10. Alternatively or additionally, server 400 can comprise a cloud-based server. Server 400 can include processing unit 410 shown, which can be configured to perform one or more functions of system 10, such as one or more functions described herein. Processing unit 410 can include one or more algorithms, algorithm 415. Processing unit 410 can comprise a memory (not shown) storing instructions for performing algorithm 415. Server 400 can be configured to receive and store various forms of data, such as: image data, diagnostic data, planning data and/or outcome data described herein, data 420. In some embodiments, data 420 can comprise data collected from multiple patients (e.g. multiple patients treated with system 10), such as data collected during and/or after clinical procedures where image data was collected from the patient via system 10. For example, image data can be collected via imaging probe 100, recorded by processing unit 310 of console 300, and sent to server 400 for analysis. In some embodiments, console 300 and server 400 can communicate over a network, for example, a wide area network such as the Internet. Alternatively or additionally, system 10 can include a virtual private network (VPN) through which various devices of system 10 transfer data.

As described herein, the one or more functions of system 10 performed by processing unit 310 and/or 410 can be performed by either or both processing units. For example, in some embodiments, image data is collected and preprocessed by processing unit 310 of console 300. The preprocessed image data can then be transferred to server 400, where the image data is further processed. The processed image data can then be transferred back to console 300 to be displayed to the operator (e.g. via GUI 353). In some embodiments, a first set of one or more images (“image” herein) that is based on a first set of image data (e.g. an image processed locally via processing unit 310) is displayed to the operator following the collection of the image data (e.g. in near-real-time), and a second image based on the first set of image data (e.g. an image processed remotely via processing unit 410) is displayed to the operator subsequently (e.g. when the first image was displayed while the second image was processed).

In some embodiments, algorithm 315 is configured to adjust (e.g. automatically and/or semi-automatically adjust) one or more operational parameters of system 10, such as an operational parameter of console 300, imaging probe 100 and/or a delivery catheter 80. Additionally or alternatively, algorithm 315 can be configured to adjust an operational parameter of a separate device, such as injector 20 and/or implant delivery device 30 described herein. In some embodiments, algorithm 315 is configured to adjust an operational parameter based on one or more sensor signals, such as a sensor signal provided by a sensor-based functional element of the present inventive concepts as described herein. Algorithm 315 can be configured to adjust (e.g. automatically adjust and/or recommend the adjustment of) an operational parameter selected from the group consisting of: a rotational parameter such as rotational velocity of optical core 110 and/or optical assembly 115; a retraction parameter of shaft 120 and/or optical assembly 115 such as retraction velocity, distance, start position, end position and/or retraction initiation timing (e.g. when retraction is initiated); a position parameter such as position of optical assembly 115; a line spacing parameter such as lines per frame; an image display parameter such as a scaling of display size to vessel diameter; an imaging probe 100 configuration parameter; an injectate 21 parameter such as a saline to contrast ratio configured to determine an appropriate index of refraction; a light source 325 parameter such as power delivered and/or frequency of light delivered; and combinations of one or more of these. In some embodiments, algorithm 315 is configured to adjust (e.g. automatically adjust and/or recommend the adjustment of) a retraction parameter such as a parameter triggering the initiation of the pullback, such as a pullback that is initiated based on a parameter selected from the group consisting of: lumen flushing (the lumen proximate optical assembly 115 has been sufficiently cleared of blood or other matter that would interfere with image creation); an indicator signal is received from injector 20 (e.g. a signal indicating sufficient flushing fluid has been delivered); a change in image data collected (e.g. a change in an image is detected, based on the image data collected, that correlates to proper evacuation of blood from around optical assembly 115); and combinations of one or more of these. In some embodiments, algorithm 315 is configured to adjust a system 10 configuration parameter related to imaging probe 100, such as when algorithm 315 identifies (e.g. automatically identifies via an RF or other embedded ID) the attached imaging probe 100 and adjusts a system 10 parameter, such as an optical path length parameter, a dispersion parameter, a catheter-type parameter, an “enabled-feature” parameter (e.g. a parameter that locks and/or unlocks the use of a feature of system 10), a calibration parameter (such as an optical length to physical length conversion parameter), and/or other parameter as listed above. In some embodiments, console 300 is configured to record one or more metrics associated with the performance of imaging probe 100, such as a brightness score. These metrics can be encoded onto probe 100 during use (e.g. encoded into an onboard memory of probe 100, such as onto a writeable RFID tag). Additionally or alternatively, fault information can be encoded onto probe 100 (e.g. written onto an RFID tag), such as when a fault occurs and/or is detected by system 10. For example, fault information can include date and time of image loss, and/or other diagnostic information, such as inability to calibrate.

In some embodiments, algorithm 315 is configured to trigger the initiation of a pullback based on a time-gated parameter. In some embodiments, a T-wave trigger (e.g. provided by a separate device) can be provided to console 300 to begin pullback when the low-motion portion of the heart cycle is detected. As an alternative to a T-wave trigger, or in addition to it, motion patterns (e.g. relative motion patterns) can be tracked (e.g. using angiography) between one or more portions (e.g. components or other features) of imaging probe 100 and relatively stable (e.g. non-moving) portions of the patient's anatomy (e.g. ribs, sternum and/or spinal column).

In some embodiments, system 10 comprises one, two or more calibration instruments, calibration tool 50 shown. Calibration tool 50 can be configured to detect and/or analyze light, such as is described in reference to FIG. 5 herein. Calibration tool 50 can comprise one or more robotic manipulators, such as to manipulate one or more tools and/or components of probe 100 during a closed-loop calibration process.

When a console 300 of system 10 is first installed at a clinical site (e.g. a catheter lab), a calibration routine can be performed (e.g. performed by and/or using calibration tool 50) 50), such as a calibration routine used to establish the latency between an angiographic system (e.g. second imaging device 15) of the clinical site and other components of system 10. Essentially, an imaging probe 100 is provided, an angiographic system at the clinical site is engaged, and an angiographic image feed is provided to console 300 (e.g. using any standard video connection, analog or digital). Angiographic system-provided video frames are registered according to a clock of console 300, which is used as a reference time frame. A pullback (e.g. in a patient or in a non-patient simulation mode) of imaging probe 100 is initiated (also coordinated by the console 300 clock) and captured by angiography (e.g. device 15). A trained operator (e.g. a clinician and/or technician) can review the angiographic image frames and designate the first frame in which motion was detected. This process establishes the associated latency according to the console 300 clock. The motion detection can also be automated, for example using a neural network or other algorithm (e.g. of algorithm 315 and/or 415) trained to recognize imaging probe 100 movement (e.g. movement of a marker band of imaging probe 100) under angiography.

In some embodiments, a calibration procedure (e.g. as performed by and/or using calibration tool 50) configured to establish the latency between an angiographic system (e.g. second imaging device 15) and other components of system 10, and an imaging procedure performed during relatively low motion of a heart cycle, includes the following steps. In a first step, angiography is initiated once probe 100 has been inserted into the patient and deployed into the target anatomy. In a second step, system 10 analyzes the relative motion between one or more portions of imaging probe 100 (e.g. motion of a marker band or other imaging probe 100 portion which follows the beating heart of the patient) and more stable features in the image, such as images of the sternum or spinal column. Once a cardiac rhythm has been established and the low motion portion identified (typically 5-10 heart cycles are used for this analysis, which can be velocity vector analysis, neural network analysis, and the like), an indicator is provided, and a system 10 “metronome” is started. System 10 can reference the output of the metronome, such as at the time that radiopaque flushing material is injected to clear the blood from the target area to be imaged, since the one or more portions of imaging probe 100 (e.g. one or more marker bands) can become radio-invisible during this flushing period (e.g. radiopaque portions of probe 100 cannot be differentiated from the flushing material). In an alternative embodiment, a non-radiopaque flushing material can be used (e.g. dextran). In a third step, flushing is started, such as by an operator or in an automated way controlled by system 10. The flushing continues over several heart cycles, such as 3-5 heart cycles. In a fourth step, clearing of the vessel to be imaged is detected by system 10 analyzing one or more of the images produced by system 10. In a fifth step, at the low motion part of the metronome (e.g. a predicted low motion portion of the heart cycle), and accounting for the latency between system 10 components and the angiographic system previously established, a pullback starts. In some embodiments, the pullback will finish in about one-half of a heart cycle or less, such as to cause capture of all or a portion of image data to remain within the low motion portion of the heart cycle. System 10 can be configured to provide a pullback speed of at least 50 mm/sec, such as at least 100 mm/sec, or 200 mm/sec. In a sixth step, the pullback sequence of images, which include minimal motion artifact, can be provided to the operator and/or used for: CFD calculations (described herein), implant (e.g. stent) length measurements, and the like. The use of image capture during low motion, as described herein, avoids or at least reduces errors associated with motion artifacts, notably longitudinal motion artifacts.

In some embodiments, algorithms 315 and/or 415 (“algorithm 315/415” herein) are configured to perform various image processing of the image data produced by system 10. Algorithm 315/415 can comprise one, two, or more artificial intelligence algorithms configured to perform the various image processing and/or other calculations, as described herein. For example, algorithm 315/415 can comprise neural networks implemented using features of DDNet and/or UNet methodologies, such as features tailored for the processing and segmentation of intravascular image data. In some embodiments, algorithm 315/415 can comprise one or more algorithms of similar configuration as the algorithm described herein in reference to FIG. 2.

Algorithm 315/415 can comprise one or more algorithms that are configured to perform one or more image processing applications selected from the group consisting of: an image quality assessment; procedural device segmentation, such as guide catheter and/or guidewire segmentation; implant segmentation, such as segmentation of endovascular implants such as stents and/or flow-diverters; lumen segmentation, such as segmentation of a vascular lumen; segmentation of side-branches; tissue characterization, such as a characterization of atherosclerotic versus normal; detection of thrombus; and combinations of these.

In some embodiments, algorithm 315/415 comprises various signal and/or image processing algorithms configured to process and/or analyze image data collected by system 10. Using these algorithms, system 10 can be configured to perform an automated quantification of one or more parameters, such as one or more patient parameters (e.g. parameters relating to the health of the patient), one or more image parameters (e.g. parameters relating to the quality of the image data), one or more treatment parameters (e.g. parameters relating to the clinical efficacy and/or technical proficiency of a treatment performed), and combinations of these. For example, system 10 can comprise a metric (e.g. a variable), data metric 525 shown, which can comprise a calculated result that is calculated using, and/or otherwise based on an analysis (e.g. a mathematical analysis) of these various parameters.

Data metric 525 can represent a quantification of the quality of image data, such as a quantification determined by an automated process of system 10. In some embodiments, data metric 525 can comprise a “confidence metric” that represents the quality of the results of an image processing step (e.g. a segmentation process). A data metric 525 comprising a confidence metric can represent a calculated level of accuracy of the image data as determined by system 10 (i.e. the level of “confidence” with which an operator of system 10 can have in the data being presented). In some embodiments, when data metric 525 comprises a confidence metric below a first threshold value (e.g. a value indicating low confidence), system 10 alerts the operator, such as via an indicator displayed to the operator via GUI 353. Additionally or alternatively, system 10 can be configured to not display any image data if a confidence metric related to that image data is below a second threshold value (e.g. a value indicating less confidence than the first threshold value). In some embodiments, system 10 can be configured to display to the operator an alert (e.g. a low confidence data warning) and/or prompt the operator to allow the display of the low confidence image data.

In some embodiments, data metric 525 comprises a quantification of one or more characteristics (e.g. level of apposition or amount of protrusion) describing the interaction between the patient's anatomy and a treatment device (e.g. implant 31) that has been implanted in the patient. For example, system 10 can be configured to analyze image data collected prior to, during, and/or after implantation of an implant, and to determine one or more values of data metric 525 that represent (e.g. correspond to) the interaction between the implant and patient tissue (e.g. the vessel wall, the ostium of one or more side-branches, and/or the neck of one or more aneurysms).

In some embodiments, data metric 525 comprises a metric relating to the healing proximate an implantation site, for example when system 10 is used to collect image data from an implantation site in a follow-up procedure, such as a procedure performed at least one month, at least six months, or at least one year from the implant procedure.

In some embodiments, data metric 525 comprises a metric relating to a predicted outcome of an interventional procedure, such as a metric whose value is calculated and/or updated during the interventional procedure, after the interventional procedure, or both. For example, data metric 525 can be used to provide guidance to the operator by indicating the predicted outcomes of intended (e.g. future) and/or already performed interventions (e.g. based on an analysis of the potential efficacy of the intervention), such as interventions configured to treat brain aneurysms and/or ischemic strokes. For example, the mesh density of a flow diverter covering the neck of an aneurysm can be estimated by system 10 (e.g. based on automated image processing described herein). The mesh density can be used to predict the outcome of the intervention (e.g. long-term dissolution of the aneurysm). Additionally or alternatively, the geometry of the mesh can be used to estimate the angle of optical assembly 115 relative to the surface of the mesh, and to correct the mesh density accordingly. For example, in a bend, the light exiting optical assembly 115 (e.g. the beam of light being transmitted from optical assembly 115) may be along an oblique angle to the mesh surface normal. In this scenario, the mesh pattern will be elongated in the plane of incidence (e.g. the plane defined by the surface normal and the light beam) according to the angle of the light beam. Correcting this elongation to achieve a symmetric pattern can provide the angle of the light beam, and this angle information can be used by system 10 to correct the calculated density of the mesh.

In some embodiments, data metric 525 comprises a metric that informs (e.g. its value is used to recommend or otherwise inform) the patient's clinician to potentially perform an additional (e.g. second) therapeutic procedure on the patient, such as to optimize or at least improve the therapeutic treatment in which at least a first procedure (e.g. an interventional procedure) has already been performed. The additional therapeutic procedure can comprise an interventional procedure selected from the group consisting of: an adjustment to a device (e.g. treatment device 16) implanted in the patient in a previous procedure, such as an adjustment comprising a repositioning, expansion, contraction, and/or other adjustment to the implant; implantation of a device (e.g. device 16) into the patient, whether or not a previous device had been implanted in the patient; a vessel dilation procedure; an atherectomy procedure and/or other procedure in which occlusive material is removed; a coiling or other procedure in which undesired space within the vascular system is occluded; a drug-delivery procedure; and combinations of these.

In some embodiments, system 10 can identify if a myocardial bridge exists over a portion of an imaged vessel. For example, system 10 can automatically detect the presence of a myocardial bridge (e.g. via algorithm 315/415), and/or the data presented to the operator of system 10 can indicate the presence of a myocardial bridge (e.g. such that the operator can draw conclusions based on the data presented). In some embodiments, image data can be collected by system 10 during a pullback procedure in which imaging probe 100 is retracted at a speed in which multiple heart cycles are captured during the pullback, such that the strain on the imaged vessel (e.g. strain caused by motion of the heart) can be analyzed throughout the heart cycle. In some embodiments, system 10 is configured to identify a myocardial bridge by analyzing image data to detect an artifact in the image data indicating the presence of a myocardial bride (e.g. a signature artifact, similar to an echolucent “halo” that can be seen when imaging a myocardial bridge using intravascular ultrasound).

In some embodiments, system 10 is configured to quantify the quality of image data, such as a quantification determined by an automated process of system 10, such as is described herein. In some embodiments, if the quality of the image data is below a threshold, one or more analytic processes of system 10 (e.g. image analysis described herein) may be disabled, such that the process is not performed on poor quality image data. For example, if image data is analyzed and it is determined (e.g., by system 10 and/or an operator of system 10) that optical assembly 115 started and/or ended within a stent during a pullback procedure, system 10 can be configured to disable subsequent CFD or other calculations described herein based on that poor image data. In some embodiments, system 10 can assess the quality of a purge procedure based on the quality of the image data. For example, system 10 can assess image quality to identify blood ingress into delivery catheter 80, and indicate the need to purge. This analysis can be used for providing feedback to the user in real-time during imaging, such as by displaying a warning message (e.g. “purge catheter”). Similarly, after an image acquisition is completed, system 10 can analyze the image data and display a warning to the user if catheter purge was incomplete. In some embodiments, system 10 can analyze image data to identify blood residuals in the lumen, and to display a warning to the user as well as indicate to the user areas where blood clearance is incomplete. If blood clearance is incomplete in the region of high interest for CFD calculation (such as obscuring frame of reference or a stenosis), a warning can be provided about insufficient image quality for a CFD calculation.

In some embodiments, system 10 is configured to perform various computational fluid dynamics (CFD) and/or optical flow ratio (OFR) calculations using high-resolution image data (e.g. OCT image data) to accurately simulate blood flow in a stenosed artery (e.g. a coronary artery), and to estimate pressure drops through one or more lesions, such as is described herein. These methods provide the user (e.g. interventionalists) with a combined and simultaneous measurement of arterial anatomy and vessel hemodynamic conditions (e.g. “Physio-Anatomy”) in high-resolution, that can be used to better characterize and diagnose the significance of stenosed coronary arteries pre-intervention, as well as following intervention (e.g. post-intervention). This information can be used to provide informed guidance and/or to optimize intervention steps as detailed herein.

Traditional clinical practice is limited to the use of either intravascular imaging (e.g. OCT or IVUS imaging) or physiology measurements (e.g. FFR, iFR, RFR, etc.) at one time, as imaging and physiology measurements can only be acquired using separate instruments (e.g. single purpose catheters). System 10 can be configured to enable capture (e.g. in a single “pullback acquisition”) of both vessel anatomy and physiology. This combined solution has the key advantage of providing intrinsically co-registered anatomy and physiology data (e.g. data captured with a single device), that can be used to better plan and optimize coronary interventions than any of these tools alone.

In some embodiments, CFD simulations performed by system 10 are designed to closely simulate hyperemic conditions, for example as it is done for the acquisition of fractional flow reserve data using a pressure wire. Alternatively or additionally, CFD methods can be used to simulate non-hyperemic conditions, for example similar to the way iFR or RFR catheters are used to collect vessel hemodynamic data. FFR devices typically make a single FFR measurement from a single location distal to all lesions. Using CFD methods of system 10 described herein, blood flow and pressure drops can be more easily evaluated for the entire coronary segment imaged with OCT.

Traditional FFR methodologies suffer from a major limitation due to lesion crosstalk. For example in the case of serial lesions, an FFR acquisition is unable to discern the individual contribution of each lesion. The CFD methods of system 10 described herein will be able to determine the contribution of each lesion, indicating which of the imaged lesions is more significant and which to treat.

System 10 can be configured to achieve a CFD simulation and pressure drop evaluation of a whole arterial segment (e.g. 100 mm or more) in a few seconds (e.g. less than 20 sec) using a simplified quasi-2D and/or 2D solver. If compared to a “full” 3D solver (e.g. a solver configured to implement Navier Stokes equations), a quasi-2D and/or 2D solver allows for an order of magnitude or more reduced computational time, retaining sufficient accuracy for coronary pressure drop evaluation.

In some embodiments, CFD simulations heavily rely on the segmentation of image data (e.g. OCT image data). Segmentation can be obtained through traditional image processing algorithms and/or AI methodologies (e.g. machine learning, deep learning, neural network, and/or other artificial intelligence methodologies). In some embodiments, these methodologies include the various steps of Method 1000, described in reference to FIG. 5 herein, to analyze image data sets (e.g. OCT image data sets) to quantify blood flow and/or pressure drops.

In some embodiments, system 10 comprises a graphical user interface, such as GUI 353 described herein, for example in reference to FIGS. 3A-C. In some embodiments, the GUI is configured to provide the user with an easy and immediate way to obtain and use OCT images and/or simulated physiology data to diagnose coronary stenoses, plan, and optimize coronary interventions. In some embodiments, OCT-FFR “Physio-Anatomy” data can be registered to coronary angiography data to provide a comprehensive tool for interventionalists to accurately plan and guide coronary procedures. Additionally or alternatively, OCT-FFR simulations can be used to create a virtual stenting tool that allows the user of system 10 (e.g. an interventionalist) to simulate the effect of stents of different lengths and diameters over different vessel locations to optimize stent sizing and selection and devise an optimal intervention strategy.

In some embodiments, Physio-Anatomy data can be quantified (e.g. by system 10) by the means of several metrics. For example, these metrics can be used to quantify the effect of the treatment pre-intervention vs. post-intervention (e.g. a “gain” quantification).

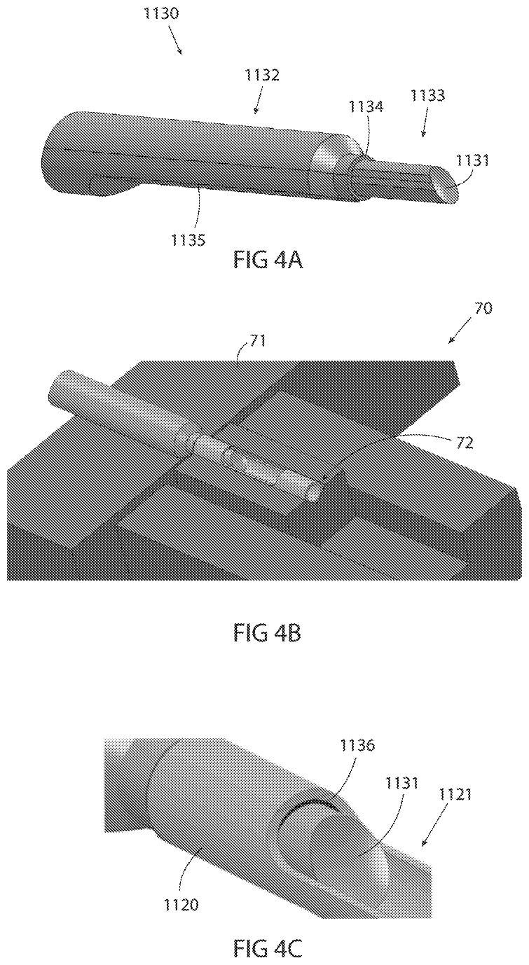

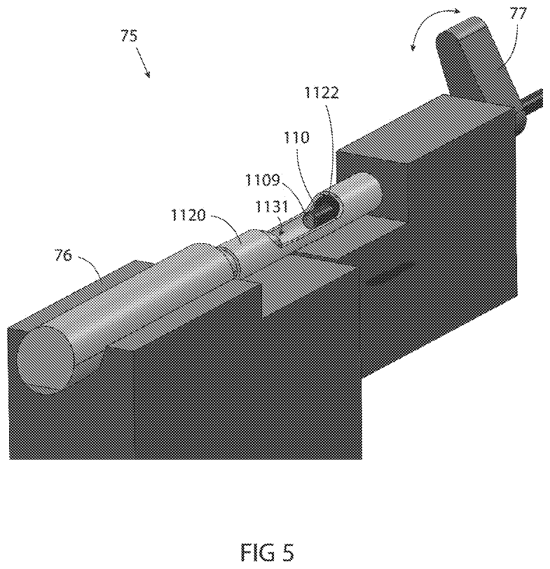

In some embodiments, system 10 is configured to ensure data quality and suitability for CFD calculations. For example, system 10 can be configured to ensure confidence of segmentation results (e.g. side-branch and/or lumen segmentation), by determining a “confidence metric”, such as is described herein. The goal of a confidence metric is to inform the user about potential images with reduced quality where segmentation results are uncertain, allowing for a quick visual review and correction (if needed). In some embodiments, system 10 can be configured to ensure that a complete pullback has been acquired, from a location distal to a lesion to the tip of the guide catheter. In some embodiments, a complete pullback can be defined as: a pullback that captured the entire disease; a pullback that did not start and/or end on a diseased vessel segment; and, if a stent is present, it is imaged in its entirety. If a pullback starts and ends on diseased vessel segments, system 10 can be configured to recover from this situation and provide an accurate CFD measurement. For example, in this scenario, system 10 can identify (e.g. via one or more methodologies described herein) healthy vessel segments and can be configured to use branching laws to estimate vessel diameters and/or areas in proximal and/or distal reference frames, for example as described in reference to FIGS. 4A-4D herein.

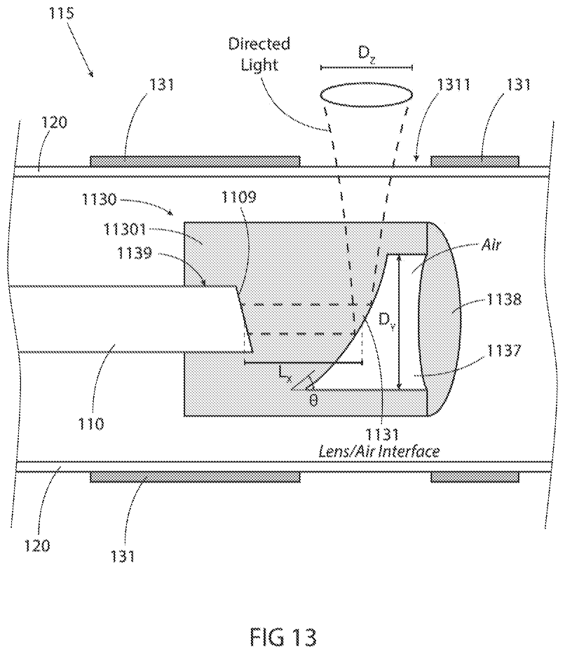

In some embodiments, system 10 is configured to perform an assessment of image quality comprising an assessment of the presence of significant blood residuals in the vessel lumen during a pullback, for example blood that obscures one or more portion of the vessel. System 10 can be configured to perform an assessment as described in reference to FIGS. 7A-7D herein. In some embodiments, system 10 is configured to assess blood that is trapped with a portion of a catheter that is configured to be imaged through (e.g. a portion of a catheter that is configured to be purged with saline before and/or during a pullback), where the trapped blood degrades the image quality. An example of incomplete catheter purging and its effects is shown in FIG. 13 described herein. One or more algorithms of system 10 can be configured to automatically detect degradation in image quality as well as the degree of quality loss, and to warn the user about the poor image quality and potential need to repeat acquisition (e.g. to repeat the pullback). In some embodiments, system 10 is configured to capture one or more angiography images. Analysis of angiography data performed by system 10 can reveal the presence of major collateral vessels. In this case, FFR and CFD calculations might be inaccurate (e.g., false low FFR in the “donor artery” and/or false high FFR in the “recipient artery”). In some embodiments, a warning message can be displayed to the user to inform about presence of collateral vessels before a CFD calculation is performed by system 10 and/or before the results are displayed by system 10 to the user.

In some embodiments, system 10 is configured to use various image processing techniques (e.g. as described herein) to help prevent incomplete and/or low-quality image data that can reduce the accuracy of CFD simulations for pressure-drop calculations. An automated determination of image data quality can warn the user of system 10 about potential issues, can help the user in correcting some issues where possible (e.g. help and/or enable the user to fix inaccurate segmentation results), and/or can indicate to the user when a new image data acquisition might be necessary. Automated assessment of data quality can warn and/or provide guidance to the user about moderate quality images and facilitate corrections. Alternatively or additionally, a severe loss of image data quality that cannot be recovered can be displayed to the user, and system 10 can provide guidance on how to improve the image quality (e.g. direct the user to better purge the catheter, and/or to better engage the coronary ostium with the guide catheter) and perform an additional image acquisition.

In some embodiments, system 10 can determine reference diameters (e.g. proximal and distal reference diameters) as well as the size of side-branches (e.g. as described in reference to FIGS. 4A-D herein) and can use this information to calculate an “ideal” and/or “reference” vessel profile to better guide intervention and/or to quantify “stent expansion”. An ideal vessel profile is a metric that can inform a more accurate stent sizing. Stent expansion is a metric that can inform additional steps to optimize stent implantation procedures.

Information collected and/or analyzed by system 10 can be used to provide various functions in a clinical environment. For example, system 10 can be used as a tool to provide training, such as training to a clinician or other user of system 10, and/or can provide equipment diagnostics information in a clinical setting, such as self-diagnostic information and/or diagnostic information related to equipment in the clinical setting that is not a part of system 10. When used in a training scenario, system 10 can be configured to perform an initial and/or periodic assessment of the user of system 10, for example by comparing determinations made by the user (e.g. based on image data gathered by system 10 and input into system 10), to determinations made by system 10 (e.g. by algorithm 315/415) based on similar data (e.g. the same data). For example, system 10 can perform an automated image assessment (e.g. to determine if blood is present during imaging, if a guide catheter is properly positioned during imaging, and/or if a catheter lumen was sufficiently purged during imaging). Based on the automated assessment, system 10 can provide feedback to the user based on the user's operation of system 10 and/or the users interpretation of the data. For example, system 10 can suggest IQ improvement, provide considerations based on the image quality assessment, and/or provide an overall pullback review.

When used in a diagnostic scenario, system 10 can perform an image quality assessment, and infer (e.g. via algorithm 315/415) from the image quality if a component of system 10 may be the cause of poor image quality. For example, system 10 can detect serviceable issues such as a failing imaging assembly 320 (e.g. from dim image data), poorly connected and/or broken connectors, and/or poor image registration (e.g. caused by NURD or other physical conditions of the catheter). In some embodiments, system 10 is configured to track the usage of various components of the system, for example the number of pullbacks for which an imaging probe 100 and/or an imaging assembly 320 has been used. In some embodiments, system 10 is configured to analyze a first set of image data collected by system 10, as well as a second set of image data from another imaging device (e.g. second imaging device 15), and to analyze (e.g. via algorithm 315/415) the image quality of the second set of image data, such as to provide a diagnostic report of the second imaging device (e.g. to determine if the second device is working properly or is in need of service and/or calibration).

In some embodiments, system 10 is configured to perform an automated review of image data gathered by the system to ensure the image quality is sufficient to perform subsequent calculations based on the image data (e.g. FFR calculations described herein). System 10 can be configured to identify various issues from image data, such as issues selected from the group consisting of: blood in the image, such as caused by inadequate blood clearing; reduced lumen wall confidence; image distortion, such as distortion caused by NURD; lack of guide catheter visualization; insufficient pullback distance, such as less than 40 mm; improper beginning and/or ending points of image data (e.g. starting and/or ending within a stent); and combinations of these.

In some embodiments, system 10 is configured to analyze image data to determine if the patient meets any exclusion criteria (e.g. such that the patient would be excluded from further treatment and/or diagnosis by system 10). Exclusion criteria identified by system 10 can include: presence of a chronic total occlusion (CTO) in the target vessel; severe diffuse disease in the target vessel (e.g. defined as the presence of diffuse, serial gross luminal irregularities present in the majority of the coronary tree); presence of myocardial bridge (MB); target lesion involves the Left Main (e.g. stenosis >50%); an artifact observed in a pre-PCI OCT image for the target lesion or in the event of multiple target lesions; an artifact observed in a pre-PCI OCT image for all target lesions; presence of a target lesion that will need to go through any preparation (including but not limited to balloon dilatation, atherectomy, and the like) prior to pre-PCI OCT imaging and physiology measurement, or in case of multiple target lesions, all target lesions that will need to go through any preparation (including but not limited to balloon dilatation, atherectomy, etc.) prior to pre-PCI OCT imaging and physiology measurement; target lesion and/or significant coronary artery disease (CAD) beyond 60 mm from coronary ostium (e.g. inability to image lesion with OCT in one pullback); incorrect and/or otherwise unsuccessful catheter purge and/or contrast flush; presence of plaque rupture and/or intravascular hematoma in target vessel (visual % diameter stenosis≥40%); and combinations of these.

In some embodiments, system 10 is configured to analyze angiography image data to identify a vessel within which imaging probe 100 is positioned (e.g. which vessel image data collected by system 10 represents). In some embodiments, one or more algorithms of system 10 (e.g. algorithm 315/415) is modified based on the vessel being imaged (e.g. automatically modified based on the identification of the vessel being imaged from angiography image data). In some embodiments, system 10 is configured to perform motion correction of OCT image data by analyzing velocity vectors of angiographic image data collected simultaneously with the OCT image data.

In some embodiments, the image processing methodologies of system 10 described herein are configured to automatically perform a process selected from the group consisting of: identify normal and diseased segments of an imaged vessel; identify ideal reference frames for vessel sizing (e.g. to avoid placing a reference segment in a diseased area); optimize scaling laws by avoiding diseased segments as reference diameters; optimize vessel size estimation; and combinations of these.

Referring now to FIGS. 2, 2a, and 2b a perspective view of an imaging probe, and two sectional views of a portion of the imaging probe, are illustrated, consistent with the present inventive concepts. Imaging probe 100 of FIG. 2 can be of similar construction and arrangement to, and include similar components as, imaging probe 100 described in reference to FIG. 1 and otherwise herein. In some embodiments, imaging probe 100 includes a shaft, outer shaft 140 as shown, including a lumen 141 within which another shaft, shaft 120 as shown, is rotatably and/or slidingly positioned. Connector assembly 150 can include a first housing, shell 151, that is fixedly attached to the proximal end of outer shaft 140, and a second housing, inner shell 152, that is fixedly attached to the proximal end of shaft 120. An optical connector, connector 153 shown, can be fixedly and optically attached to the proximal end of optical core 110, such that the rotation of connector 153 causes a corresponding rotation of optical core 110. Connector 153 can be rotatably connected to inner shell 152, such that connector 153 can rotate relative to inner shell 152 (e.g. when attached to rotation assembly 210, not shown but described herein in reference to FIG. 1). In some embodiments, PIU 200 (not shown but also described herein in reference to FIG. 1) is configured to rotate optical core 110. PIU 200 can be further configured to also retract optical core 110, shaft 120, or both (e.g. in unison) relative to outer shaft 140. For example, connector assembly 150, including shell 151 and inner shell 152 can be constructed and arranged to operably connect to a mating connector of PIU 200, into which inner shell 152 and connector 153 are retracted, thus shaft 120 can be retracted relative to outer shaft 140 by PIU 200. PIU 200 can be configured to attach to and retract inner shell 151 and shaft 120 without rotating inner shell 151 and/or shaft 120 (e.g. to rotate connector 153 and shaft 110 relative to inner shell 152 and shaft 120, respectively.

In some embodiments, outer shaft 140 includes an assembly that provides a location for shaft 120 to safely buckle when under compression, buckle assembly 145. Buckle assembly 145 can include an elongate structure, housing 146, positioned between two segments of shaft 140. Housing 146 can include one or more chambers, for example chamber 147 shown. In some embodiments, shaft 120 extends through a first segment of shaft 140, through chamber 147, and continues through the second segment of shaft 140, such that shaft 120 is unsupported within chamber 147, providing a “buckle point” for shaft 120 (e.g. a location in which shaft 120 can safely buckle). Should shaft 120 encounter resistance (e.g. become elastically compressed) as it is advanced through shaft 140, chamber 147 allows shaft 120 to safely buckle, such as to prevent or at least limit the likelihood that shaft 120 (and/or optical core 110) punctures and/or otherwise undesirably exits shaft 140 (e.g. due to an undesired rapid advance of shaft 120 due to built-up compression within shaft 120).

In some embodiments, at least a distal portion of shaft 140 comprises an optically transparent segment, such as window 142 shown. In some embodiments, window 142 comprises polyetheretherketone (PEEK), or another optically transparent material (e.g. at least partially transparent to light at the wavelength produced by light source 325). Shaft 120 can be configured to be translated within shaft 140 such that optical assembly 115 translates within window 142 (e.g. within all or a portion of window 142). In some embodiments, the distal portion of optical core 110, including optical assembly 115, is positioned at a location within shaft 140 and beyond the distal end of shaft 120 (as shown in FIG. 2B). In some embodiments, gel 118 surrounds a distal portion of optical core 110 within shaft 120. Probe 100 can be configured such that gel 118 does not exit shaft 120 into shaft 140 (e.g. gel 118 remains within shaft 120), for example, such that gel 118 does not enter the optical path of optical assembly 115. In some embodiments, outer shaft 140 includes a rapid exchange tip, tip 1191 as shown.

Referring now to FIGS. 3 and 3A, a perspective view and a sectional view of an optical assembly are illustrated, consistent with the present inventive concepts. Imaging probe 100 including optical assembly 115 of FIGS. 3 and 3A can be of similar construction and arrangement to optical assembly 115 described in reference to FIG. 1 and otherwise herein. Optical assembly 115 can include an elongate structure, housing 1120, that is constructed and arranged to maintain the position of various components of optical assembly 115, such as are described herein. For example, as shown, housing 1120 can be fixedly attached to the distal portion of optical core 110, as well as to a reflecting element, reflector 1130. Distal end 1109 of optical core 110 can be rotationally aligned and axially positioned relative to a reflecting surface, surface 1131, of reflector 1130, such as an angled reflecting surface (e.g. a surface that is not orthogonal to the axis of reflector 1130). In some embodiments, reflector 1130 comprises an injection molded component. In some embodiments, reflective surface 1131 comprises a shape that is created during a molding process. Alternatively or additionally, reflective surface 1131 can comprise a machined surface (e.g. a surface that is formed in a machining process after the body of reflector 1130 is molded or otherwise formed) and/or a surface that is 3D printed (e.g. when reflector 1130 is 3D printed). In some embodiments, surface 1131 of reflector 1130 comprises a reflective coating, such as a gold plating. In some embodiments, optical core 110 is fixedly attached to housing 1120, such as with adhesive 1122 (as shown) and/or with another attachment element (e.g. a heat shrink tube). In some embodiments, adhesive 1122 comprises a glue configured to be cured with ultraviolet light. In some embodiments, housing 1120 comprises a material configured to enhance the visibility of optical assembly 115, for example a radiopaque material configured to be imaged using fluoroscopy, such that an operator of system 10 can identify the location of optical assembly 115 in a fluoroscopic image.

Surface 1131 of reflector 1130, and/or distal end 1109 of optical core 110 can each comprise a geometry configured to direct the path of light emitted from optical core 110 to tissue and/or a geometry configured to direct light reflected from tissue into optical core 110 (either or both, “direct” light herein), as described herein. In some embodiments, distal end 1109 comprises an angled surface, as shown (e.g. a surface that is not orthogonal to the axis of core 110), such as when optical core 110 comprises an angle-cleaved fiber and/or a fiber including an angled polish. Housing 1120 can comprise an opening, opening 1121 shown. Surface 1131 and distal end 1109 can be configured to direct light through opening 1121. Alternatively or additionally, housing 1120 can comprise an optically transparent material (e.g. an optically transparent window segment) through which light can be directed. In some embodiments, optical assembly 115 is positioned within shaft 140 and is surrounded by air (e.g. surrounded by a gas such as atmospheric air), such that the directed light travels through air between distal end 1109 and the wall of outer shaft 140. The air gap between distal end 1109 and reflector 1130 allows the light to expand, and then the concave surface of reflector 1130 allows the light to be collimated. Optical assembly 115 can be positioned in air within air (e.g. atmospheric air) within shaft, eliminating the need to purge shaft 140.

In some embodiments, and as shown in FIG. 3A, optical assembly 115 extends beyond the distal end of shaft 120 and is rotatably and slidingly positioned within the window 142 portion of outer shaft 140, such as is described in reference to FIG. 2B herein. As shown in FIG. 3A, outer shaft 140 comprises inner diameter D1 and outer diameter D4, optical assembly 115 (e.g. lens housing 1120) comprises outer diameter D3, lens housing 1120 comprises inner diameter D2, shaft 120 comprises inner diameter D5, and optical assembly 115 comprises length L1. In some embodiments, diameter D3 of optical assembly 115 is greater than inner diameter D5 of shaft 120 (e.g. as optical assembly 115 is positioned distal to the distal end of shaft 120).

In some embodiments, imaging probe 100 comprises a 1.8 F shaft, for example when the dimensions of optical assembly 110 comprise approximately the following dimensions: D1 can comprise a diameter between 0.010″ and 0.045″ (e.g. D1 can comprise a diameter of at least 0.010″ and/or a diameter of no more than 0.045″), such as a diameter of approximately 0.018″, D2 can comprise a diameter between 0.005″ and 0.040″, such as a diameter of approximately 0.012″, D3 can comprise a diameter between 0.005″ and 0.042″, such as a diameter of approximately 0.014″, and D4 can comprise a diameter between 0.012″ and 0.050″, such as a diameter of approximately 0.023″. In some embodiments, L1 comprises a length between 0.040″ and 0.500″ (e.g. a length of at least 0.040″ and/or a length of no more than 0.500″).

Referring now to FIGS. 4A-C, perspective views of a reflector, an assembly tool, and an optical assembly are illustrated, consistent with the present inventive concepts. In some embodiments, reflector 1130 includes a projection, handle 1132, which is configured to be used during a manufacturing process, and eventually removed (e.g. removed and discarded). Handle 1132 extends from a functional portion of reflector 1130, insert 1133, which can be inserted into housing 1120 during the manufacturing of optical assembly 115. Surface 1131 is positioned on the proximal end of insert 1133 as shown. A distal portion of insert 1133 can include a radial projection, shoulder 1134, that radially extends to a diameter greater than the inner diameter of housing 1120, such as to control (e.g. limit or establish) the depth that insert 1133 can be inserted into housing 1120. Handle 1132 can include one or more alignment features, feature 1135, which can comprise a flat surface as shown. In some embodiments, system 10 includes an assembly tool, jig 70. Feature 1135 can comprise a flat surface that slidingly mates with jig 70, such as a mating flat surface of jig 70, surface 71 shown in FIG. 4B. In some embodiments, alignment feature 1135 comprises a keyed or other shape that is configured to align handle 1132 with a mating shape of assembly jig 70. Alignment feature 1135 can be rotationally aligned with surface 1131, such that when handle 1132 is aligned with assembly jig 70, surface 1131 is oriented to be properly mated with housing 1120. Assembly jig 70 can include an alignment element, recess 72, which can be constructed and arranged to slidingly receive housing 1120 such that housing 1120 can be slid over insert 1133, such as to position surface 1131 within opening 1121. In some embodiments, recess 72 is constructed and arranged to rotationally align housing 1120 with respect to surface 71 (such that opening 1121 is rotationally aligned with surface 1131, such that light is directed through opening 1121 via surface 1131). Alternatively or additionally, opening 1121 can be configured to be manually (e.g. “by eye”) aligned with surface 1131. In some embodiments, insert 1133 is fixedly attached to housing 1120, such as with adhesive 1136 as shown in FIG. 4C.

Referring now to FIG. 5, a perspective view of an assembly tool is illustrated, consistent with the present inventive concepts. System 10 can include an assembly tool, jig 75, constructed and arranged to assist a manufacturing assembler to rotationally align optical core 110 (e.g. distal end 1109 of optical core 110) with surface 1131 of reflector 1130 within housing 1120. Alignment feature 1135 of reflector 1130 can mate with jig 75, such as with a flat surface 76 of jig 75, such that reflector 1130 and housing 1120 are rotationally fixed relative to jig 75. Jig 75 can include a rotational tool, torquer 77, that removably attaches to optical core 110, such as to assist a manufacturing assembler in rotating optical core 110 to align distal end 1109 with surface 1131 of reflector 1130. Once aligned, optical core 110 can be affixed (e.g. glued) to housing 1120 with adhesive 1122. In manufacturing, after insert 1133 is affixed to housing 1120, and the assembly of insert 1133 and housing 1120 is then affixed to the distal portion of optical core 110, handle 1132 of reflector 1130 can be removed (e.g. cut off and discarded), such that optical assembly 115 is in a fully assembled state, as shown in FIG. 3.

In some embodiments, during the manufacturing process, and while optical core 110 is aligned with housing 1120, core 110 can be attached to a light source (e.g. the proximal end of core 110 is attached to a light source, such as a light source as described herein), and the light exiting distal end 1109 is reflected by surface 1131 of reflector 1130. The reflected light (i.e. the light emitted from optical assembly 115) can be observed, such as when system 10 comprises a calibration device (e.g. calibration tool 50 described in reference to FIG. 1), which can be configured to analyze the emitted light, and the alignment of core 110 can be adjusted to optimize the emitted light, as described herein. In some embodiments, torquer 77 comprises a robotically controlled component (e.g. via a robotic manipulator of calibration tool 50), such that the axial and rotational position of distal end 1109 of core 110 can be adjusted in a closed loop fashion (e.g. as calibration tool 50 analyzes the emitted light and robotically or otherwise adjusts the alignment to optimize the light emitted from optical assembly 115).

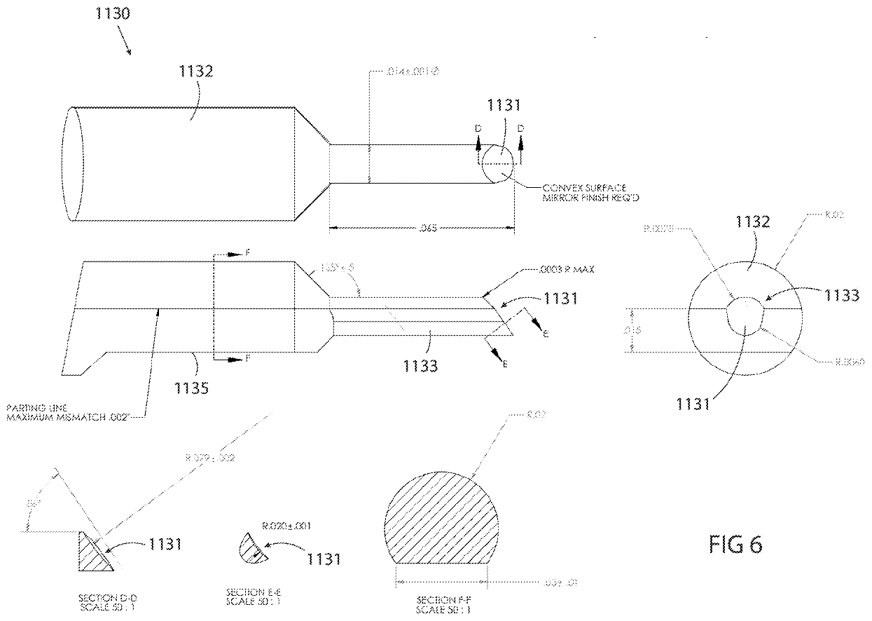

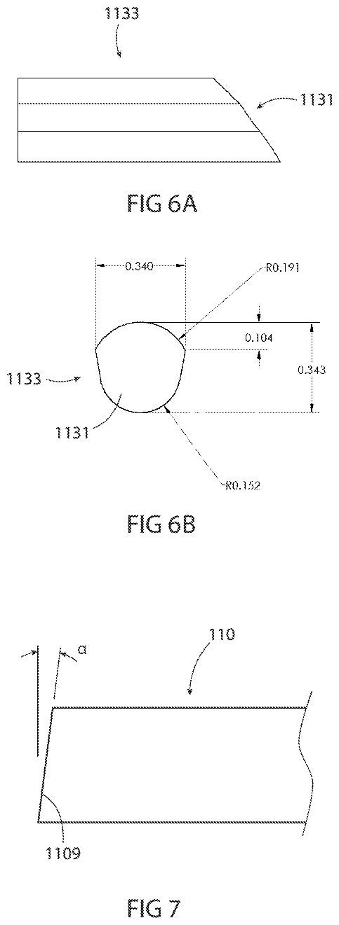

Referring now to FIG. 6, various views of an embodiment of a reflector are illustrated, consistent with the present inventive concepts. Reflector 1130, including handle 1132 and insert 1133, can comprise a component constructed and arranged as shown in FIG. 6. Referring additionally to FIGS. 6A and 6B, an end view and a side view, respectively, of a portion of a reflector are illustrated, consistent with the present inventive concepts. Insert 1133 of FIGS. 6A and 6B can be of similar construction and arrangement to insert 1133 described in reference to FIG. 3A and otherwise herein.

Referring now to FIG. 7, a side view of the distal end of an optical fiber is illustrated, consistent with the present inventive concepts. Optical core 110 of FIG. 7 can be of similar construction and arrangement to optical core 110 described in reference to FIG. 1 and otherwise herein. In some embodiments, distal end 1109 of optical core 110 comprises an angled surface, such as a surface with an angle a as shown. Angle a can comprise an angle between 0° and 20° (e.g. an angle of at least 0° and/or an angle of no more than 20°), such as between 8° and 12° (e.g. an angle of at least 8° and/or an angle of no more than 12°), such as an angle of approximately 8°.

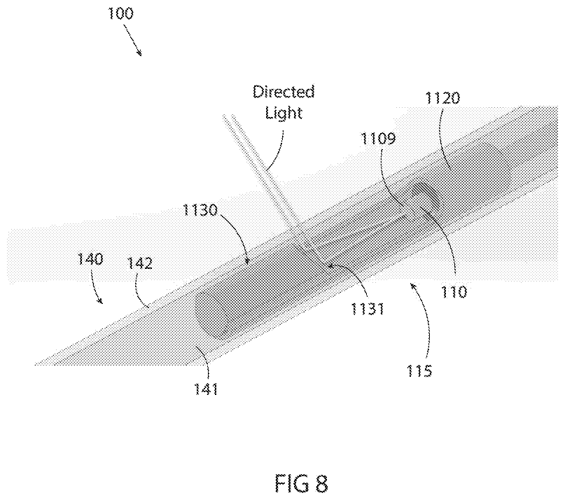

Referring now to FIG. 8, a partially transparent, perspective view of the distal portion of an imaging probe is illustrated, consistent with the present inventive concepts. Imaging probe 100 of FIG. 8 can be of similar construction and arrangement as imaging probe 100 described in reference to FIG. 1 and otherwise herein. Optical assembly 115 can be positioned on the distal end of optical core 110 and it can be located within lumen 141 of outer shaft 140, as shown. As described herein, light provided by light source 325, not shown, to optical assembly 115, exits distal end 1109 of optical core 110 and reflects off of surface 1131 of reflector 1130, as shown. The directed light reflects off of reflector 1130 and extends through the window 142 (e.g. through the wall of the portion of shaft 140 comprising window 142). In some embodiments, surface 1131 of reflector 1130 comprises a gold plating, as described herein.

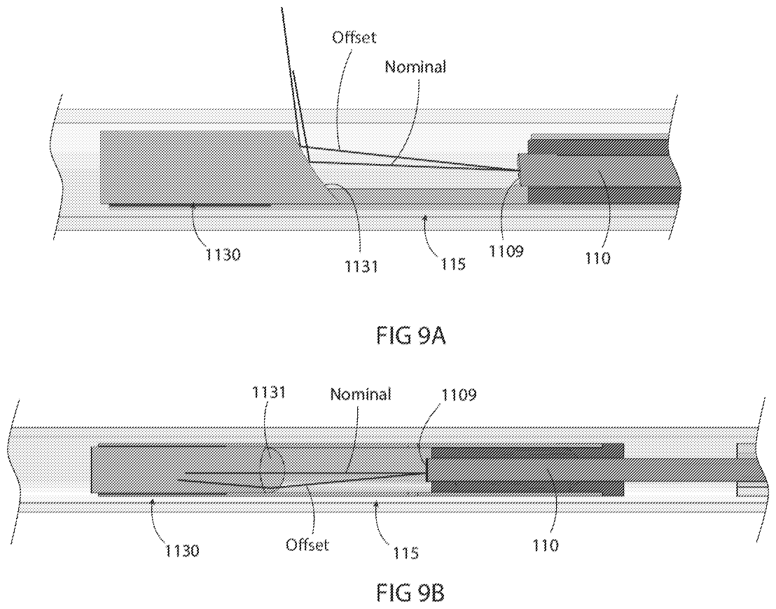

Referring now to FIGS. 9A and 9B, side sectional and top sectional views, respectively, of the distal end of an imaging probe are illustrated, consistent with the present inventive concepts. FIGS. 9A and 9B show nominal and offset light paths (e.g. non-nominal light paths caused by a misalignment between reflector 1130 and distal end 1109 of optical core 110). The offset light paths shown represent approximately a 50 μm offset. In some embodiments, the geometry (e.g. curvature) of surface 1131 is configured to prevent such offsets (e.g. offsets caused in a manufacturing process) from adversely affecting the focus of light leaving optical core 110. Alternatively or additionally, the geometry (e.g. curvature) of surface 1131 is configured to avoid the creation of unwanted stray light.

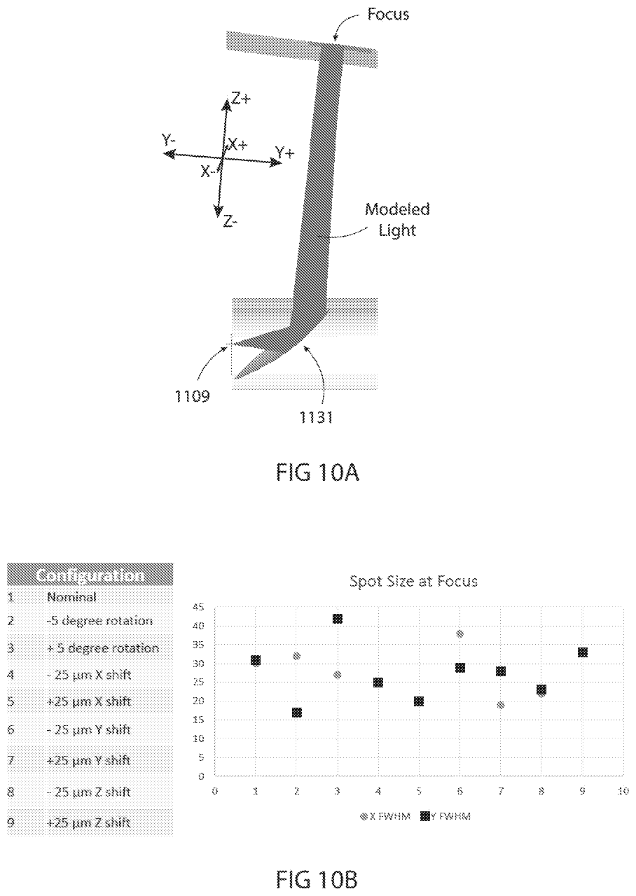

Referring now to FIGS. 10A and 10B, optical modeling images and charts of results are illustrated, consistent with the present inventive concepts. Applicant has performed simulations including optical modeling of the performance of optical assembly 115 described herein. FIG. 10A shows the path of modeled light exiting distal end 1109 of optical core 110 that reflects off of surface 1131 to a focal spot. Modeling showed X1/e²@focus=30μ, Y1/e²@focus=31μ, X1/e²<35 0-3.2 mm, Y1/e²<35 0.7-4.4 mm. Modeling showed that at the focus point, the X and Y diameters of the focal spot are 30 μm and 31 μm, respectively. This result indicates good symmetry in X and Y. Furthermore, modeling showed that the X diameter remained below 35 μm up to 3.2 mm, and the Y diameter remained below 35 μm up to 4.4 mm. The X and Y dimensions of the beam are perpendicular to the axis of the beam (approximately 80° from the axis of probe 100), and are primarily orthogonal to and parallel to the axis of probe 100, respectively, as shown. Modeling also showed a coupling efficiency of 77%. Coupling showed a focus depth of from 0 mm and 4.4 mm. FIG. 10B shows a chart of the X and Y variance of the size of the focal spot for various alignment shifts between optical core 110 and reflector 1130. These results indicate that the construction and arrangement to optical assembly 115 sufficiently compensates for distortion caused by window 142 (e.g. a 1.8 Fr sheath), and that focus can be maintained over a full range of potential manufacturing tolerances.

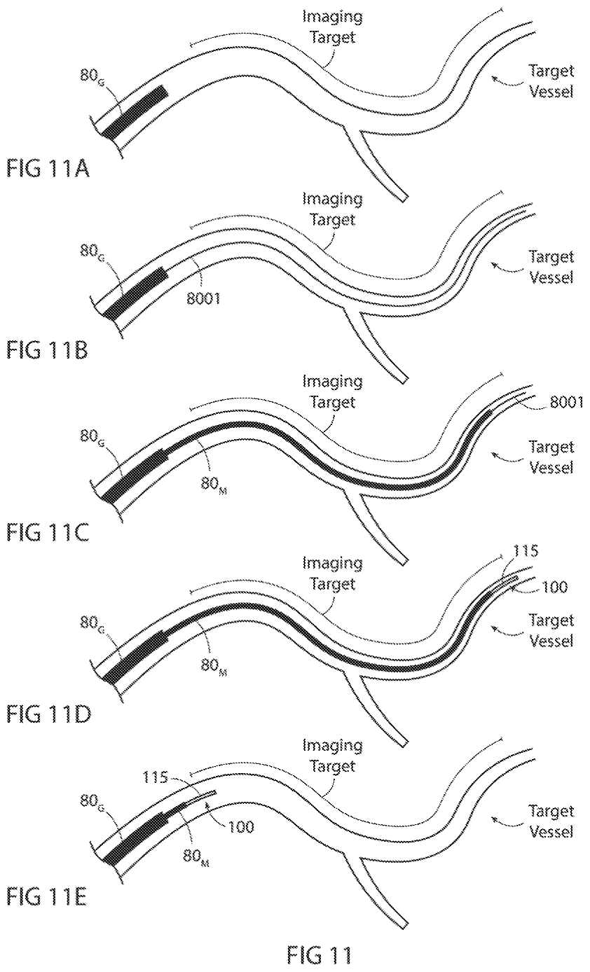

Referring now to FIGS. 11A-E, anatomic sectional side views of the steps of an imaging method are illustrated, consistent with the present inventive concepts. In FIG. 11A, a first delivery catheter, guide catheter 80G, has been advanced into a vessel (e.g. a blood vessel within the patient's brain or other head location), proximate an imaging target (e.g. proximate the proximal end of a length of a target vessel to be imaged). In FIG. 11B, a guide wire, wire 8001, has been advanced through guide catheter 80G, and through the target vessel to the distal end of the imaging target (e.g. extending at least to the distal end of the imaging target). In some embodiments, wire 8001 is inserted prior to guide catheter 80G, which can be inserted over wire 8001.

In FIG. 11C, a second delivery catheter, micro catheter 80M has been advanced within guide catheter 80G and over wire 8001 to the distal portion of the imaging target. In FIG. 11D, wire 8001 has been removed, and an imaging probe, such as imaging probe 100 described herein, has been advanced through micro catheter 80M. In some embodiments, at least a distal portion of optical probe 100, including imaging assembly 115, extends beyond the distal end of micro catheter 80M, as shown. Alternatively, imaging assembly 115 may remain within micro catheter 80M, for example when the distal end of micro catheter 80M comprises a transparent segment through which imaging probe 100 can image the target vessel (e.g. a transparent segment through which imaging probe can transmit and received light). In some embodiments, imaging probe 100 is advanced to a position where the distal end of micro catheter 80M is approximately collocated with, and/or beyond the distal end of imaging probe 100, and micro catheter 80M can be subsequently retracted to expose imaging assembly 115 (e.g. while imaging probe 100 is held in place). Alternatively, imaging probe 100 can be configured to be safely advanced beyond the distal end of micro catheter 80M, for example when imaging probe 100 comprises a spring tip or other feature, geometry, and/or flexibility configured to prevent or at least limit the likelihood of a traumatic injury to the target vessel during advancement beyond the distal end of micro catheter 80M.

In FIG. 11E, micro catheter 80M and imaging probe 100 have been retracted in unison during a pullback procedure. For example, retraction assembly 220 (not shown but described in reference to FIG. 1) can be configured to retract micro catheter 80M and imaging probe 100 in unison.

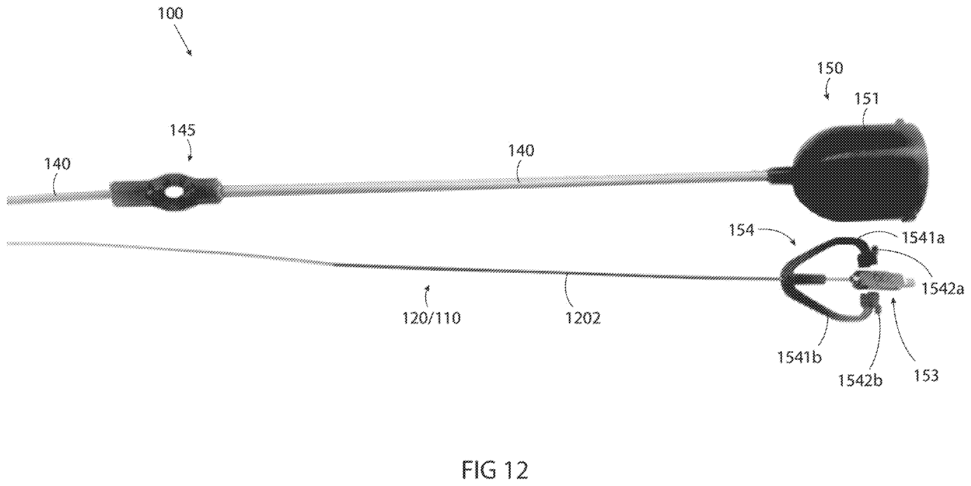

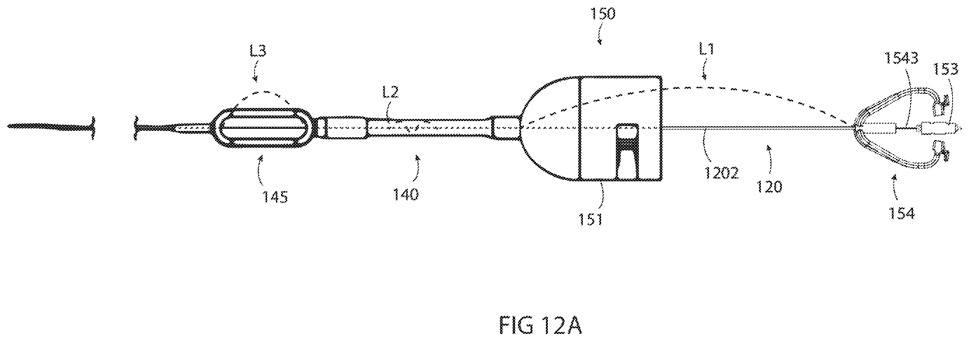

Referring now to FIG. 12, a side view of a portion of an imaging probe is illustrated, consistent with the present inventive concepts. Imaging probe 100 of FIG. 12 can be of similar construction and arrangement as imaging probe 100 described in reference to FIG. 1 and otherwise herein. Shaft 120, surrounding optical core 110, is shown positioned next to outer shaft 140. Components of connector assembly 150 are located on the proximal ends of shafts 140 and 120. Shaft 120 is constructed and arranged to be slidingly received within shaft 140, such as is described herein. Shaft 140 can include buckle assembly 145 that provides a location for shaft 120 to safely buckle when under compression (e.g. when shaft 120 is positioned within shaft 140 and buckle assembly 145), as described herein. Connector 150 can include a housing, shell 151 (e.g. described hereinabove), that is fixedly attached to the proximal end of shaft 140, as shown.

In some embodiments, connector 150 includes a stabilizing assembly, wishbone 154 shown. Wishbone 154 can be fixedly attached to the proximal end of shaft 120, and wishbone 154 can be configured to frictionally engage an optical connector, connector 153 shown, which is positioned on the proximal end of optical core 110. Optical core 110 and connector 153 can be rotatable relative to shaft 120 and wishbone 154. In some embodiments, wishbone 154 frictionally engages (e.g. lockingly engages) connector 153, such as to prevent connector 153 from moving (e.g. rotating and/or moving axially with respect to shaft 120). In some embodiments, wishbone 154 is rotated (e.g. rotated relative to shell 151) to engage and/or release connector 153, for example as described herebelow. Wishbone 154 can comprise one or more elongate members, arms 1541a, b shown. Arms 1541a, b can be resiliently biased inward, towards connector 153, such that in a resting position, arms 1541a, b oppose connector 153 and prevent undesired rotation of connector 153 (e.g. to maintain a desired rotational orientation between shell 151 and connector 153). Alternatively, arms 1541a, b can be resiliently biased outward, away from connector 153, such that in a resting position, arms 1541a, b are positioned away from connector 153, allowing free rotation of connector 153.

Connector 150 can be constructed and arranged to operably attach to PIU 200, not shown but described herein. PIU 200 can comprise a connector configured to operably attach to (e.g. optically, electrically, and/or mechanically attach to) connector 150. For example, connector 153 can connect to rotation assembly 210 of PIU 200, such that rotation assembly 210 can rotate connector 153 and optical core 110. In some embodiments, PIU 200 is configured to apply a force to separate arms 1541a, b from connector 153 (e.g. to “open” wishbone 154) to allow connector 153 to rotate. In some embodiments, attaching shell 151 to PIU 200 causes wishbone 154 to open. For example, shell 151 can be configured to rotate (e.g. to rotate approximately 90°) to attach to PIU 200 and/or to open wishbone 154. In some embodiments, shell 151 comprises an elliptical inner shape, such that by rotating wishbone 154 relative to shell 151, arms 1541a, b of wishbone 154 rotate from the minor axis of shell 151 into the major axis of shell 151, allowing arms 1541a, b to expand into an open position (e.g. when arms 1541a, b are biased in an open position). In some embodiments, wishbone 154 is configured to attach to retraction assembly 220 of PIU 200, such that wishbone 154 and connector 153, which are attached to shaft 120 and optical core 110, respectively, are retracted in unison (e.g. during a pullback procedure). In some embodiments, arms 1541a, b include one or more features (e.g. geometric features) configured to engage retraction assembly 220, such as hooks 1542a, b shown.

In some embodiments, shaft 120 includes a reinforced portion, portion 1202. Portion 1202 can comprise a length at least as long as a maximum pullback distance of retraction assembly 220 (e.g. a maximum pullback distance made available by system 10), such as a distance of at least 15 cm. In some embodiments, portion 1202 comprises a stiffness greater than the stiffness of shaft 120 distal to portion 1202. Portion 1202 can be configured to avoid buckling, such as to avoid buckling when under a compressive load of 1.5 pounds or more. In some embodiments, the rotating portion of connector 150 (e.g. connector 153) comprises a mass of less than 3 g, such as less than 2 g, such as approximately 1.1 g, such as to reduce the angular momentum of connector 153.

Referring additionally to FIG. 12A, a side view of a portion of an imaging probe is illustrated, consistent with the present inventive concepts. During the advancement of shaft 120 into outer shaft 140 (e.g. after a pullback procedure), binding of the two shafts may cause shaft 120 to buckle. Under compression, shaft 120 may buckle at locations L1, L2, and/or L3, as shown. The force required to buckle at location L1, between wishbone 154 and shaft 140, changes as shaft 120 is advanced, and the unsupported length of portion 1202 shortens. Buckling of shaft 120 at location L2, within shaft 140, does not prevent additional force (e.g. axial force) from being applied to shaft 120 distal to location L2 (e.g. additional force which could cause damage to probe 100 and/or injury to the patient due to undesired advancement of probe 100). Buckling at location L3, via buckle assembly 145, can safely relieve compressive forces caused by binding of shaft 120 distal to assembly 145 and prevent any associated undesired advancement (e.g. unintentional and/or uncontrolled advancement) of the distal end of shaft 120.

In some embodiments, wishbone 154 includes a hollow tube, tube 1543, which extends between wishbone 154 and connector 153, as shown. Optical core 110 can extend from the proximal end of shaft 120, through tube 1543, to connector 153. Tube 1543 can be configured to provide support to (e.g. prevent buckling of) optical core 110 between shaft 120 and connector 153. In some embodiments, tube 1543 comprises a thermoplastic material, such as PEEK. In some embodiments, wishbone 154 comprises a polycarbonate material.

Referring now to FIG. 13, a sectional view of the distal end of an imaging probe is illustrated, consistent with the present inventive concepts. Imaging probe 100 of FIG. 13 can be of similar construction and arrangement as imaging probe 100 described in reference to FIG. 1 and otherwise herein. FIG. 13 shows an embodiment of optical assembly 115 including a reflector (e.g., reflector 1130 shown) configured to provide a lens/air interface constructed and arranged to reflect directed light from optical core 110 towards tissue. In some embodiments, reflector 1130 is positioned on the distal end of optical core 110, as shown. Reflector 1130 can comprise a body portion, body 11301, that includes a recess that forms a chamber-portion of reflector 1130, such as chamber 1137, each as shown. The proximal surface of chamber 1137 can comprise a reflective surface, reflective surface 1131 shown (e.g., such that the proximal face of reflective surface 1131 provides a reflective interface for internal reflection). In some embodiments, reflector 1130 includes a cap or other sealing element, seal 1138 shown, that encloses chamber 1137, preventing ingress of fluids and/or other materials into chamber 1137. Air and/or other one or more gases that is sealed within chamber 1137 provides a lens/air interface for reflective surface 1131, such that reflector 1130 is configured to reflect light from core 110 using “Total Internal Reflection” (TIR). In some embodiments seal 1138 comprises an adhesive, such as a UV glue. In some embodiments, reflective surface 1131 comprises a coating, such as a sputter coating and/or an evaporation deposition coating, for example a gold and/or an aluminum coating. The coating can be applied to all or a portion of reflector 1130. For example, the coating can be applied only to reflective surface 1131, such as when reflector 1130 is held by a fixture during the coating process, where the fixture prevents the coating from being undesirably applied to other portions of reflector 1130. In some embodiments, distal end 1109 comprises an angled distal face, as shown, such as a distal face at an angle of less than 20°, 10°, and/or 5°. Alternatively or additionally, distal end 1109 can comprise a flat (e.g., not angled) face, such as a face that is perpendicular to the axis of optical core 110. In some embodiments, reflector 1130 and optical core 110 can comprise index-matched materials, for example two materials comprising matching indices of reflection. In some embodiments, reflector 1130 is manufactured onto optical core 110 using an over-molding process. Alternatively or additionally, reflector 1130 can be adhered to optical core 110, such as when reflector 1130 comprises a machined, molded, and/or otherwise constructed part that is adhered onto optical core 110, for example in a manufacturing assembly process. Reflector 1130 can include a mating portion that physically attaches to optical core 110, such as recess 1139 shown. Recess 1139 can slidingly receive the distal portion of optical core 110, such as when optical core 110 is inserted into and adhered to reflector 1130. In some embodiments, the geometry of recess 1139 approximates (e.g., closely matches) the geometry of the distal end of optical core 110. For example, if the distal end of optical core 110 comprises an angled face, the distal surface of recess 1139 can comprise an angled surface that matches the angle of optical core 110.

Reflective surface 1131 can comprise an angle, angle θ shown, where θ is measured relative to the longitudinal axis of optical core 110. Angle θ can comprise an angle of at least 45°, such as at least 47°. Chamber 1137 can comprise diameter D_{y} shown. Diameter D_{y} can comprise a diameter of at least 0.2 mm, such as no more than 0.8 mm. Reflective surface 1131 can be positioned a distance Lₓ from distal end 1109 of optical core 110, as shown. Distance Lₓ can comprise a distance of at least 0.3 mm, such as no more than 1.40 mm. In some embodiments, optical assembly 115 is configured to image a spot size comprising a diameter D_{z}. Diameter D_{z} can be approximately 30 μm.

In some embodiments, imaging probe 100 includes one or more markers, such as marker 131 shown. In some embodiments, marker 131 includes one or more openings, such as opening 1311 shown, where opening 1311 allows directed light from optical core 110 to exit shaft 120 (e.g., without being blocked by marker 131). In some embodiments, reflector 1130 comprises a polycarbonate material. In some embodiments, a top (relative to the page) portion of reflector 1130 (e.g., the portion of reflector 1130 through which the directed light exits and/or enters reflector 1130) comprises a flat surface, such as when reflector 1130 comprises a cylindrical geometry with a polished flat top portion.

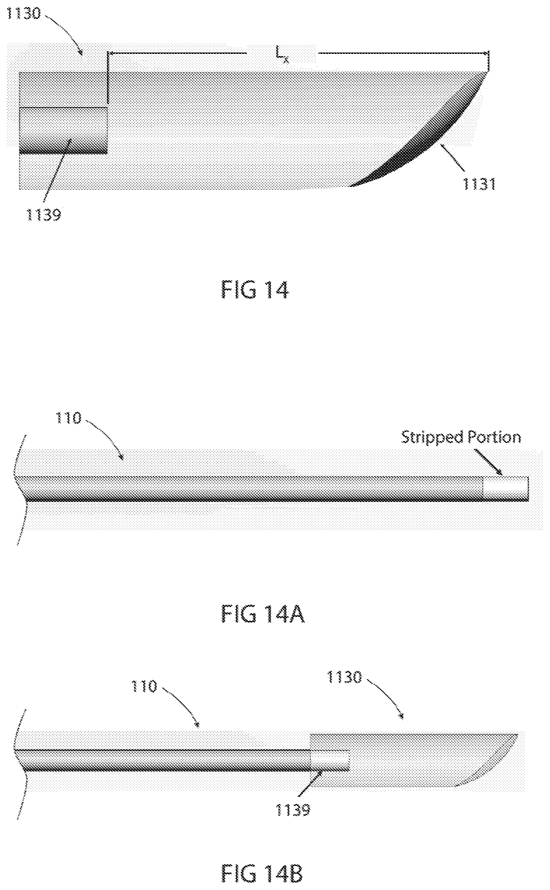

Referring now to FIGS. 14, 14A, and 14B, side views of a lens assembly, the distal end of an optical fiber, and the lens assembly positioned on the distal end of the optical fiber are illustrated, respectively, consistent with the present inventive concepts. Imaging probe 100 of FIGS. 14, 14A and 14B can be of similar construction and arrangement as imaging probe 100 described in reference to FIG. 1 and otherwise herein. FIG. 14 shows an embodiment of a molded reflector, reflector 1130. FIGS. 14A and 14B illustrate steps of preparing a fiber for attachment to a reflector, and assembly of the reflector onto the prepared fiber. Reflector 1130 can include a recess, recess 1139 shown, that slidingly receives the distal portion of optical core 110. In some embodiments, optical core 110 includes one or more coatings, such as a protective coating. A portion of a coating can be removed (e.g., stripped and/or otherwise removed) from the distal portion of optical core 110 prior to insertion into recess 1139, for example as shown in FIG. 14A. In some embodiments, reflective surface 1131 includes a lens/air interface, for example when reflector 1130 is positioned in air within shaft 120 of probe 110 (not shown but described herein), such that reflector 1130 provides total internal reflection. In some embodiments, for example when reflector 1130 provides total internal reflection, reflective surface 1131 does not include a reflective coating.

In some embodiments, reflector 1130 comprises a diameter of at least 200 μm. In some embodiments, reflective surface 1131 includes a geometry with one or more curvatures, such as a compound curvature configured to correct for astigmatism caused by the curvature of shaft 120 and/or the outer surface of reflector 1130. Reflective surface 1131 can comprise a nominal polish angle of approximately 40° relative to the axis of optical core 110 (e.g., a polish angle configured to provide total internal reflection). Reflector 1130 can comprise a length Lx from distal end 1109 of optical core 110 to the distal end of reflector 1130, as shown. The length Lₓ can comprise a length of at least 0.3 mm, a length of no more than 1.40 mm, or both.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. An imaging system for a patient comprising:
an imaging probe, comprising:
a first elongate shaft comprising a proximal end, a distal portion, a distal end, and a lumen extending at least between the proximal end and the distal portion;
a second elongate shaft comprising a proximal end, a distal end, and a lumen extending between the proximal end and the distal end, wherein at least a portion of the second elongate shaft is positioned within the lumen of the first elongate shaft;
a rotatable optical core comprising a proximal end and a distal end, wherein at least a portion of the rotatable optical core is positioned within the lumen of the second elongate shaft; and
an optical assembly positioned proximate the distal end of the rotatable optical core, the optical assembly configured to direct light to tissue to be imaged and to collect reflected light from the tissue to be imaged; and
an imaging assembly constructed and arranged to optically couple to the imaging probe, wherein the imaging assembly is configured to emit light into the imaging probe and to receive the reflected light collected by the optical assembly.

2.-27. (canceled)
